SEQUENCE LISTING (20 sequences)

SEQ ID NO: 1 - protein, 147 aa, Hordeum vulgare
MASDHRRFVLSGAVLLSVLAVAAATLESVKDECQPGVDFPHNPLATCHTYVIKRVCGRGPSRPMLVKERCCRELAAVPDHCRCEALRILMDGVRTPEGRVVEGRLGDRRDCPREEQRAFAATLVTAAECNLSSVQEPGVRLVLLADG

SEQ ID NO: 2 - DNA, 1286 bp, Hordeum vulgare
ttattggacaccaaatgtatcataaacttgttttttcaccgacaaaatattgctcctccatttcgcattaaaattgtcaagcatgcttgcaacagtaacacgaacattcataaaaaaaatattttttaagaaaacatttactatttttttgttactattcatctgggagcatgtgcttccggaagccaaaatgccccttccaatatgccccgtgtaaaagaaaccccttctttcctaaaaatatatatcatcgtccgtcatgatacgtttatgtattcaacgaaaaatattttcgcatgtcaccaaaaatgttttatattacacaagtgaacaaatatgataaactccctcgtgttaactattttttctgtgaaataaaaggatgacaatcaaaacaaaaatgtagactgtaaacaaagaaaacattatttcctagaaataaaaaaaaagattagagggatatgtattgtcgaaacacatgaggactagaacaaaagaaaaagggaaatgagaaggaaaaaaggggtaaccattacccaaagaaaacagaaagtaaactagacgtgtcgaagggaaacggagtttgcaggggcgttccaaattcagttgcaagaacctccaaataaacgccaacaagaaagaaatgagcattacttgcgcgctttgcactcttatctctagcatctcccgatacatacatacatgtagcctagctgcagatcttgaatagctattcttgcccaccaggccaagagattgaaccaacgaccaataaactagtatcaacaatggcatccgaccatcgtcgcttcgtcctctccggcgccgtcttgctctcggtcctcgccgtcgccgccgccaccctggagagcgtcaaggacgagtgccaaccaggggtggacttcccgcataacccgttagccacctgccacacctacgtgataaaacgggtctgcggccgcggtcccagccggcccatgctggtgaaggagcggtgctgccgggagctggcggccgtcccggatcactgccggtgcgaggcgctgcgcatcctcatggacggggtgcgcacgccggagggccgcgtggttgagggacggctcggtgacaggcgtgactgcccgagggaggagcagagggcgttcgccgccacgcttgtcacggcggcggagtgcaacctatcgtccgtccaggagccgggagtacgcttggtgctactggcagatggatgacgatcgaaatgcgccaaggtaatgaagcggagtactgtatacagaataaaagta

SEQ ID NO: 3 - protein, 147 aa, Hordeum vulgare
MASDHRRFVLSGAVLLSVLAVAAATLESVKDECQPGVDFPHNPLATCHTYVIKRVCGRGSSRPMLVKERCCRELAAVPDHCRCEALRILMDGVRTPEGRVVEGRLGDRRDCPREEQRAFAATLVTAAECNLSSVQEPGVRLVLLADG

SEQ ID NO: 4 - protein, 147 aa, Hordeum vulgare
MASDHRRFVLSGAVLLSVLAVAAATLESVKDECQPGVDFPHNPLATCHTYVIKRVCGRGLSRPMLVKERCCRELAAVPDHCRCEALRILMDGVRTPEGRVVEGRLGDRRDCPREEQRAFAATLVTAAECNLSSVQEPGVRLVLLADG

SEQ ID NO: 5 - protein, 147 aa, Hordeum vulgare
MASDHRRFVLSGAVLLSVLAVAAATLESVKDECQPGVDFPHNPLATCHTYVIKRVCGRGPSRPMLMKERCCRELAAVPDHCRCEALRILMDGVRTPEGRVVEGRLGDRRDCPREEQRAFAATLVTAAECNLSSVQEPGVRLVLLADG

SEQ ID NO: 6 - protein, 147 aa, Hordeum vulgare
MASDHRRFVLSGAVLLSVLAVAAATLESVKDECQPGVDFPHNPLATCHTYVIKRVCGRGPSRPMLVKKRCCRELAAVPDHCRCEALRILMDGVRTPEGRVVEGRLGDRRDCPREEQRAFAATLVTAAECNLSSVQEPGVRLVLLADG

SEQ ID NO: 7 - protein, 905 aa, Hordeum vulgare
MAVGETGASVSAAEAEAEATQAFMPDARAYWVTSDLIAWNVGELEAQSVCLYASRAAAMSLSPSNGGIQGYDSKVELQPESAGLPETVTQKFPFISSYRAFKVPSSVDVASLVKCQLVVASFGADGKHVDVTGLQLPGVLDDMFAYTGPLGAVFSEDSVSLHLWAPTAQGVSVCFFDGPAGPALETVQLKESNGVWSVTGPREWENRYYLYEVDVYHPTKAQVLKCLAGDPYTRSLSANGARTWLVDINNETLKPASWDELADEKPKLDSFSDITIYELHIRDFSAHDGTVDSDSRGGFRAFAYQASAGMEHLRKLSDAGLTHVHLLPSFHFAGVDDIKSNWKFVDECELATFPPGSDMQQAAVVAIQEEDPYNWGYNPVLWGVPKGSYASDPDGPSRIIEYRQMVQALNRIGLCVVMDVVYNHLDSSGPCGISSVLDKIVPGYYVRRDTNGQIENSAAMNNTASEHFMVDRLIVDDLLNWAVNYKVDGFRFDLMGHIMKRTMMRAKSALQSLTTDAHGVDGSKIYLYGEGWDFAEVARNQRGINGSQLNMSGTGIGSFNDRIRDAINGGNPFGNPLQQGFNTGLFLEPNGFYQGNEADTRRSLATYADQIQIGLAGNLRDYVLISHTGEAKKGSEIHTFDGLPVGYTASPIETINYVSAHDNETLFDVISVKTPMILSVDERCRINHLASSMMALSQGIPFFHAGDEILRSKSIDRDSYNSGDWFNKLDFTYETNNWGVGLPPSEKNEDNWPLMKPRLENPSFKPAKGHILAALDSFVDILKIRYSSPLFRLSTANDIKQRVRFHNTGPSLVPGVIVMGIEDARGESPEMAQLDTNFSYVVTVFNVCPHEVSMDIPALASMGFELHPVQVNSSDTLVRKSAYEAATGRFTVPGRTVSVFVEPRC

SEQ ID NO: 8 - DNA, 2718 bp, Hordeum vulgare
atggcggtcggggagaccggcgcctccgtctccgcagccgaggccgaggccgaggccacccaggcgttcatgccggacgccagggcgtactgggtgacgagcgacctcatcgcctggaacgtcggcgagctggaagcgcagtccgtctgcctgtacgccagcagagccgccgcgatgagcctctcgccgtcgaatggcggcatccaaggctacgactccaaggttgagctgcaaccggagagcgccgggctcccggaaaccgtgacccagaagttccctttcatcagcagttacagagcattcaaggtcccgagctctgtcgacgtcgccagccttgtgaaatgccaactggtcgtcgcttctttcggcgctgacgggaaacacgtagatgttactggactgcaattacccggcgtgctggatgatatgttcgcatacacgggaccgctcggtgcggttttcagcgaggactctgtgagcctgcacctttgggctcctacagcacagggcgtgagcgtgtgcttctttgatggtccagcaggccctgcgctagagacggtgcagctcaaggagtcaaatggtgtttggagtgtcactggaccaagagagtgggaaaaccggtactatttgtatgaagtcgacgtgtatcatccaactaaggcgcaggttctgaaatgtttagctggtgacccttatactagaagcctttctgcaaatggagcgcgtacctggttggttgacattaacaatgagacattgaagccggcttcctgggatgaattggctgatgagaagccaaaacttgattccttctctgacataaccatctatgaattgcacattcgtgattttagcgcccacgatggcacagtggacagtgactctcgtggaggatttcgtgcatttgcatatcaggcctcggcaggaatggagcacctacggaaattatctgatgctggtttgactcatgtgcatttgttgccaagctttcattttgctggcgttgacgacattaagagcaactggaaatttgtcgatgagtgtgaactagcaacattccctccagggtcagatatgcaacaagcagcagtagtagctattcaggaagaggacccttataattgggggtataaccctgtgctctggggggttccaaaaggaagctatgcaagtgaccctgatggcccgagtcgaattattgaatatcgtcagatggtccaggccctcaatcgcataggtctttgtgttgtcatggatgttgtatacaatcatctagactcaagtggcccctgcggtatcagctcagtgcttgacaagattgttcctgggtactatgttagaagggatactaatggccagattgagaacagtgcagctatgaacaatacagcaagtgagcatttcatggttgataggttaatcgtggatgaccttttgaactgggcagtaaactacaaagttgacgggttcagatttgatcttatgggccatatcatgaaacgcacaatgatgagagcaaaatctgctcttcaaagccttacaacagatgcacatggagttgatggttcaaaaatatacttgtatggtgaaggatgggacttcgctgaagttgcacgcaatcaacgtggaataaatgggtcccagcttaatatgagtggaacggggattggtagcttcaatgatagaatccgggatgctattaatgggggtaatccctttggtaatccgctccagcaaggcttcaatactggtctgttcttagagccgaatgggttttatcagggcaatgaagcagataccaggcgctcgctcgctacttatgctgaccaaatacagattggactagctggtaatctgagggattatgtactaatatctcatactggagaagctaagaagggatcagaaattcacacttttgatggattaccagtaggctatactgcgtccccaatagaaacgataaactatgtttctgctcatgacaatgagactctatttgatgttatcagtgtgaagaccccaatgatcctttcagttgatgagagatgcaggataaatcatttggcctccagcatgatggcattatcccagggaatacccttcttccacgctggtgacgagatactaagatctaagtccatcgaccgagattcatataactctggtgattggtttaacaagcttgattttacctatgaaacaaacaattggggtgttgggcttcctccaagtgaaaagaacgaagataattggcccctgatgaaaccaagattggaaaatccgtcttttaaacctgcaaaaggacacattcttgctgccctagacagttttgttgacatcttgaagatcagatactcatctccactttttcgtctcagtacagcaaatgacattaagcaaagggtacgctttcacaacacagggccctccttagtcccaggtgttattgtcatgggcattgaagatgcacgaggtgagagccccgagatggctcaattagacacgaacttctcttatgtcgtaaccgtcttcaatgtgtgtccgcacgaagtgtccatggatatccccgctctcgcttcgatggggtttgaactgcatcctgtgcaggtgaattcatcagatactttggtgaggaaatcggcgtacgaggccgcgacgggcaggttcaccgtgcccggaagaaccgtgtcagtctttgtcgaacctcggtgttga

SEQ ID NO: 9 - DNA, 19 bp, Artificial sequence
  HENZ-16a forward primer
  primer_bind (1)..(19): HENZ-16a forward primer
ctacgtgataaaacgggtc

SEQ ID NO: 10 - DNA, 15 bp, Artificial sequence
  HENZ-16a Reverse primer
  primer_bind (1)..(15): HENZ-16a Reverse primer
ccgctccttcaccag

SEQ ID NO: 11 - DNA, 13 bp, Artificial sequence
  HENZ-16a FAM probe
  misc_feature (1)..(13): HENZ-16a FAM probe
cggctggaaccgc

SEQ ID NO: 12 - DNA, 13 bp, Artificial sequence
  HENZ-16a HEX probe
  misc_feature (1)..(13): HENZ-16a HEX probe
ccggctgggaccg

SEQ ID NO: 13 - DNA, 12 bp, Artificial sequence
  HENZ-18 Forward primer
  primer_bind (1)..(12): HENZ-18 Forward primer
gccgcggtccca

SEQ ID NO: 14 - DNA, 15 bp, Artificial sequence
  HENZ-18 Reverse primer
  primer_bind (1)..(15): HENZ-18 Reverse primer
cagtgatccgggacg

SEQ ID NO: 15 - DNA, 17 bp, Artificial sequence
  HENZ-18 FAM probe
  misc_feature (1)..(17): HENZ-18 FAM probe
atgctgatgaaggagcg

SEQ ID NO: 16 - DNA, 16 bp, Artificial sequence
  HENZ-18 HEX probe
  misc_feature (1)..(16): HENZ-18 HEX probe
atgctggtgaaggagc

SEQ ID NO: 17 - DNA, 12 bp, Artificial sequence
  HENZ-31 Forward primer
  primer_bind (1)..(12): HENZ-31 Forward primer
gccgcggtccca

SEQ ID NO: 18 - DNA, 15 bp, Artificial sequence
  HENZ-31 Reverse primer
  primer_bind (1)..(15): HENZ-31 Reverse primer
cagtgatccgggacg

SEQ ID NO: 19 - DNA, 16 bp, Artificial sequence
  HENZ-31 FAM probe
  misc_feature (1)..(16): HENZ-31 FAM probe
tggtgaagaagcggtg

SEQ ID NO: 20 - DNA, 15 bp, Artificial sequence
  HENZ-31 HEX probe
  misc_feature (1)..(15): HENZ-31 HEX probe
tggtgaaggagcggt

(19) United States
(12) Patent Application Publication
(10) Pub. No.: US 2023/0111237 A1
(43) Pub. Date: Apr. 13, 2023

(54) Barley Plants with High Limit Dextrinase Activity

(71) Applicant: Carlsberg A/S, Copenhagen V (DK)

(72) Inventors: Ole Olsen, Copenhagen S (DK); Finn Lok, Copenhagen V (DK); Søren Knudsen, Copenhagen V (DK); Lucia Marri, Copenhagen V (DK); Alexander Striebeck, Fredericksberg C (DK); Pai Rosager Pedas, Copenhagen V (DK); Jose Antonio Cuesta-Seijo, Valby (DK); Hanne Cecilie Thomsen, Copenhagen V (DK); Katarzyna Birch Braune, Copenhagen V (DK)

(73) Assignee: Carlsberg A/S, Copenhagen V (DK)

(21) Appl. No.: 17/905,431

(22) PCT Filed: Mar. 1, 2021
(86) PCT No.: PCT/EP2021/055053

§ 371 Date: Sep. 1, 2022

(30) Foreign Application Priority Data
Mar. 2, 2020 (EP) 20160355.2

(51) Int. Cl. C12N 15/82; C07K 14/415; C12C 1/027; C12C 7/00

(52) U.S. Cl. CPC C12N 15/8245; C07K 14/415; C12C 1/027; C12C 7/00

(57) ABSTRACT

The present invention provides barley plants, or parts thereof, having a high limit dextrinase activity. In particular, barley plants carrying a mutation in HvLDI gene are provided. Furthermore, plant products prepared from said barley plants, or parts thereof, are described as well as methods of producing the same.

### TECHNICAL FIELD

The present invention relates to the field of providing barley plants with high free limit dextrinase activity. In particular, the invention relates to barley plants having grains with a high free limit dextrinase activity. Grains of such plants are advantageous for production of barley based liquid extracts, e.g. wort, with increased amount of fermentable sugars. The invention further relates to methods for production of barley based beverages, e.g. beer, whiskey, vodka, or maltina, as well as to products prepared from barley plants of the invention.

### BACKGROUND

Germination is the initial part of the process by which a plant grows from a seed. In order to do so, the grain needs to keep control of a plethora of enzymes. Some enzymes are involved in the degradation of starch in the endosperm into maltose and glucose, which in turn serve as energy source for the plant embryo. The same process produces fermentable sugars that can be extracted from germinated grains or malt, and used by yeast to produce alcohol during brewing.

Starch is a carbohydrate made of two forms of glucose chains: The mainly linear amylose and the branched amylopectin. The linear parts of amylose and amylopectin can be degraded into fermentable sugars by different classes of amylases. However, amylases are typically incapable of degrading amylopectin around its branch points. Thus, amylase activities are insufficient in order to achieve an efficient release of fermentable sugars from starch.

Limit dextrinase (LD), a glycoside hydrolase, catalyses hydrolysis of the branch points of starch resulting in linear starch fragments thereby increasing the availability of substrate for amylases. LD specifically catalyses hydrolysis of alpha-1,6 linkages in e.g. amylopectin or in branched dextrins. The hydrolytic action of this enzyme results in the formation of linear alpha-1,4-linked glucose chains that can be extensively depolymerized to glucose and maltose by the combined action of alpha- and beta-amylases.

LD's activity is considered to be controlled at least in part by its inhibitor limit dextrinase inhibitor (LDI). LDI is thought to bind and inactivate LD.

Low levels of LD activity generally leads to a low degradation of starch, which is favorable during grain filling and allows sufficient levels of starch to build up in the grains. In brewing processes, extracts of germinated barley grains or malt are used as a substrate for yeast fermentation, and extracts containing high levels of fermentable sugars are generally desirable. Without the action of LD, the branched dextrins and amylopectin could not be fermented effectively by yeast.

It has been shown in the literature that downregulation of LDI by antisense in barley plants has a profound effect on the health of barley grains demonstrated by a lower grain weight, decreased number of starch granules per barley grain and altered starch synthesis, including enhanced amylose to amylopectin ratios, changes to amylopectin architecture, shown by altered branch chain length of amylopectin (more chains of 9 to 15 residues, but fewer long chains of 30-60 residues) and reduced levels of the small B-type starch granules (see Y. Stahl et al. 2004).

The interaction between LD and LDI has been studied using the crystal structure of the barley LD-LDI complex. In vitro binding studies of LDI and LD mutants have been performed, 4 different positions in LDI were mutated and showed modest to large increased in the K_{D} (see M Møller et al. 2015), however none of these mutants have been tested in vivo and it can therefore not be assessed whether the mutations would effect grain health, or whether the in vitro results translate to in vivo effects on fermentable sugar levels in the grains.

### SUMMARY

The objective of the present invention is to provide, a barley plant with grains having high LD activity, especially during germination and when subjected to malting, wherein said barley plants at the same time are healthy, and e.g. have yield and grain weight comparable to wild type barley plants. Such barley plants would be very useful in the production of barley/malt based beverages such as beer.

Barley plants with grains having high LD activity are useful in the production of barley/malt based beverages, such as beer, whiskey, vodka, or maltina. One advantage is that aqueous extracts, such as wort, prepared from grains and/or malt from a barley plant having high LD activity have a high content of fermentable sugars. Thus, by using grains and/or malt of the invention, the need for addition of exogenous limit dextrinase or pullulanase during mashing may be reduced or even completely abolished. Furthermore, fermenting an aqueous extract containing a high content of fermentable sugars is a benefit during brewing, since it increases the amount of beer produced per amount of grains used and increase the ABV % (alcohol by volume) pr. hectorliter per grain weight. Additionally, grains and/or malt from barley plants of the invention, having increased free Hordeum vulgare limit dextrinase (HvLD) activity are useful in malting processes, wherein the germination time is shortened as described in for example WO 2018/001882.

Surprisingly, the present invention provides barley plants carrying a mutation in the LDI gene, where the plant is healthy and have grain yield, grain size and grain amylopectin branch chain length comparable to wild type barley plants, but which at the same time have a high LD activity. Such barley plants are useful as raw material for preparing extracts with high contents of fermentable sugars.

In particular, the invention shows that certain mutations in the Hordeum vulgare limit dextrinase inhibitor (HvLDI) gene encode mutated HvLDI polypeptides, which have a reduced binding to HvLD in vitro. The reduced ability to bind HvLD results in an increase in free LD and thereby higher LD activity in vivo, which in turn results in a higher content of fermentable sugars in aqueous extracts prepared by using grains from a barley plant carrying a mutation of the present invention in HvLDI polypeptide. Importantly, at the same time no difference in grain size, grain amylopectin branch chain length and grain yield were observed in barley plants carrying a mutation of the invention in the HvLDI gene.

Thus, the present invention provides a barley plant, or part thereof, wherein said barley plant carries a mutation in the HvLDI gene, wherein said mutated HvLDI gene encodes a mutant HvLDI polypeptide, wherein the mutation is one of the following mutations
a. a missense mutation resulting in a change from a proline to a different amino acid in one or more loop regions of HvLDI, wherein the loop regions are selected from the group consisting of amino acids corresponding to position 25 to 44 and amino acids corresponding to position 56 to 62 and amino acids corresponding to position 77 to 78 and amino acids corresponding to position 91 to 111 and amino acids corresponding to position 124 to 147 of SEQ ID NO:1; or
b. a missense mutation resulting in a change from a negatively charged amino acid to a non-negatively charged amino acid in one or more alpha helix regions of wt HvLDI, wherein the alpha helix regions are selected from the group consisting of amino acids corresponding to position 45 to 55 and amino acids corresponding to position 63 to 76 and amino acids corresponding to position 79 to 90 and amino acids corresponding to position 112 to 123 of SEQ ID NO:1.

The invention further provides plant products, such as grains, malt, wort or beverages prepared from the barley plants of the invention.

Furthermore, methods for preparing malt are disclosed, wherein said methods may comprise the steps of:
a. providing grains of a barley plant of the invention;
b. steeping and germinating said grains under predetermined conditions;
c. optionally, drying said germinated grains.

Also, methods of producing an aqueous extract are disclosed, wherein said methods may comprise the steps of:
a. providing grains of a barley plant of the invention and/or malt prepared from such a barley plant;
b. preparing an aqueous extract of said grains and/or said malt, for example wort.

In addition, methods of producing a beverage are disclosed, wherein said methods may comprise the steps of:
a. providing grains of a barley plant of the invention and/or malt prepared form such barley plant and/or an aqueous extract according prepared from such barley plant and/or malt;
b. processing said aqueous extract into a beverage, e.g. by fermentation or mixing with other beverage components.

Furthermore, methods of preparing barley plants according to the invention are disclosed, wherein said method comprising the steps of:
a. providing barley grains; and
b. randomly mutagenizing said barley grains,
c. Selecting barley grains or parts thereof carrying a mutated HvLDI gene encoding a mutant HvLDI polypeptide carrying one of the following mutations
   i. a missense mutation resulting in a change from a proline to a different amino acid in one or more loop regions of HvLDI, wherein the loop regions are selected from the group consisting of amino acids corresponding to position 25 to 44 and amino acids corresponding to position 56 to 62 and amino acids corresponding to position 77 to 78 and amino acids corresponding to position 91 to 111 and amino acids corresponding to position 124 to 147 of SEQ ID NO:1; or
   ii. a missense mutation resulting in a change from a negatively charged amino acid to a non-negatively charged amino acid in one or more alpha helix regions of wt HvLDI, wherein the alpha helix regions are selected from the group consisting of amino acids corresponding to position 45 to 55 and amino acids corresponding to position 63 to 76 and amino acids corresponding to position 79 to 90 and amino acids corresponding to position 112 to 123 of SEQ ID NO:1.

### DESCRIPTION OF DRAWINGS

FIG. 1: shows the ability of wild type (wt) HvLDI or mutant HvLDI to inhibit wt HvLD activity in an in vitro assay. A) wt LDI (no mutation), B) HvLDI mutant (P60L), C) HvLDI mutant (P60S), D) HvLDI mutant (V66M) and E) HvLDI mutant (E68K). The Y-axis shows the % activity of HvLD. The X-axis shows the amount (μM) of wt or mutated HvLDI used in the in vitro assay. The potency of wt HvLDI and mutant HvLDI to inhibit recombinant expressed HvLD was assessed by the amount of chromophore released during the assay. The amount of released chromophore during the assay was compared to the amount released of fully active HvLD and used to calculate the retaining activity. Activity was plotted against the concentration of HvLDI used in the assay and the data was fitted with a sigmoidal response curve.

FIG. 2: shows hydrolytic enzyme activities in grains germinated for 72 h from mutant barley plants HENZ-16a (P60S) and HENZ-18 (V66M) as well as their control Paustian, and for mutant barley plant HENZ-31 (E68K) as well as its control Planet. Grains were germinated according to the germination protocol described in Example 3. EBC19 was included as an additional control in the experiment. A) shows total amount of alpha-amylase. B) shows total amount of beta-amylase. C) shows total amount of limit dextrinase and free limit dextrinase, as well as the ratio between free limit dextrinase and total limit dextrinase in %.

FIG. 3: shows hydrolytic enzyme activities in flex-malt malted grains from mutant barley plant HENZ-16a (P60S) and its control Paustian, and for mutant barley plant HENZ-31 (E68K) and its control Planet. The grains were malted according to the method described in Example 5. A) shows total amount of alpha-amylase. B) shows total amount of beta-amylase. C) shows total amount of limit dextrinase and free limit dextrinase, as well as the ratio between free limit dextrinase and total limit dextrinase in %.

FIG. 4: shows hydrolytic enzyme activities in VLB malted grains from mutant barley plant HENZ-16a (P60S) and its control Paustian. The grains were VLB malted according to Example 6. Pilsner malt was included as an additional control in the experiment. A) shows total amount of alpha-amylase. B) shows total amount of beta-amylase. C) shows total amount of limit dextrinase and free limit dextrinase, as well as the ratio between free limit dextrinase and total limit dextrinase in %.

FIG. 5: shows kinetic measurements in flex-malted grains. Filled circles and triangles show the amount of free limit dextrinase and total amount of limit dextrinase respectively in HENZ-16a (P60S). Empty circles and triangles show the amount of free limit dextrinase and total amount of limit dextrinase respectively in Paustian.

FIG. 6: shows sugar analysis in wort from flex-malted HENZ-16a (P60S), Paustian and EBC19. A) shows the amount (PPM) of total fermentable sugars (TFS), i.e. fructose, sucrose, glucose, maltose and maltotriose. The numbers above the bars represent the total amount of TFS. B) shows the amount of individual sugars present in the different worts. The numbers above the bars represent the % difference between HENZ-16a (P60S) and Paustian.

FIG. 7: shows sugar analysis in wort from VLB malted HENZ-16a (P60S), Paustian and EBC19, as well as pilsner malt. A) shows the amount (PPM) of total sugars (TFS), i.e. fructose, sucrose, glucose, maltose and maltotriose. The numbers just above the bars represent the total amount of TFS. Wort from VLB malted grains from HENZ-16a (P60S) contains 7.2% more TFS compared to wort from VLB malted grains from Paustian. B) shows the amount of individual sugars present in wort from VLB malted HENZ-16a (P60S) and Paustian. The numbers above the bars represent the % difference between HENZ-16a (P60S) and Paustian.

FIG. 8: A) and C) show chain length distribution analysis of amylopectin in grains from HENZ-16a (P60S) and HENZ-31, Planet and Paustian barley plants. B) and D) show peak area difference between the mutant barley plant and its control (Denmark, 2017). The HvLDI mutant barley plants and control plants were grown in neighboring plots in Denmark in the season 2017. It can be concluded that there is no significant difference in the DP between the HvLDI mutant barley plants (HENZ-16a and HENZ-31) compared to their respective controls. DP: degree of polymerization.

FIG. 9A) and C) shows chain length distribution analysis of amylopectin in grains from HENZ-16a and HENZ-31 barley plants. B) and D) show peak area difference between the mutant barley plant and its control (New Zealand 2017-18). The HvLDI mutant barley plants and control plants were grown in neighboring plots in New Zealand in the season 2017/2018. It can be concluded that there is no significant difference in the DP between the HvLDI mutant barley plants (HENZ-16a and HENZ-31) compared to their respective controls. DP: degree of polymerization.

### DETAILED DESCRIPTION

### Definitions

As used herein, “a” can mean one or more, depending on the context in which it is used.

The term “aeration” as used herein refers to supplying to a given material a gas comprising oxygen, e.g. pure oxygen or air. Aeration of an aqueous solution (e.g. water) is preferably performed by passing said gas through the water, e.g. by introducing the gas at the bottom and/or in the lower part of a container comprising the aqueous solution. Typically, the gas will diffuse through the aqueous solution and leave the aqueous solution from the top of the aqueous solution. Aeration of barley grains during air-rest may e.g. be performed by leading the gas through the bed of barley grains and/or passing a stream said gas over the surface of the bed of barley grains.

The term “amino acid” as used herein refers to a proteinogenic amino acid. Preferably, the proteinogenic amino acid is one of the 20 amino acids encoded by the standard genetic code. The IUPAC one and three letter codes are used to name amino acids.

The term “approximately” when used herein in relation to numerical values preferably means ±10%, more preferably ±5%, yet more preferably ±1%.

The term “amylose” refers to homopolymers of α-D-glucose. Amylose has a linear molecular structure, as its glucose units are almost exclusively linked by alpha-1,4-glycosidic bonds.

The term “amylopectin” refers to homopolymers of α-D-glucose. Amylopectin molecules contains frequent alpha-1,6-glucosidic linkages. These introduce branch points into the otherwise alpha-1,4-linked glucose chains resulting in clusters of parallel chains appearing in regular intervals along the molecule's axis.

The term “air rest” refers to a phase in the germination process following a phase where the grains have been soaked in water (aqueous solution). In the air rest phase water is drained from the grains and the grains are allowed to rest. Preferably the moisture of the grains is maintained above 20%, more preferably above 30%, even more preferably above 40% during this phase. In some embodiments, the grains are subjected to aeration during air rest. Preferably, humid air or wet oxygen is passed through the grains during the air rest. The temperature may be any suitable temperature, preferably the temperature is maintained between 20 and 28° C.

The term “barley” in reference to the process of making barley based beverages, such as beer, particularly when used to describe the malting process, means barley grains. In all other cases, unless otherwise specified, “barley” means the barley plant (Hordeum vulgare), including any breeding line or cultivar or variety, whereas part of a barley plant may be any part of a barley plant, for example any tissue or cells.

The term “different amino acid” covers proteinogenic amino acids, such as one or more of the 20 amino acids encoded by the standard genetic code.

The term “DP” or “degree of polymerization” as used herein indicates the number of alpha-1,4-linked glucose units in amylopectin side chains.

As used herein “fermentable sugars” refers to any sugar that a microorganism can utilize or ferment. In particular fermentable sugars are monosaccharides, disaccharides and short oligosaccharides, including but not limited to glucose, fructose, maltose, maltotriose and sucrose, which can be fermented by microorganisms, in particular yeast or lactobacteria, to produce ethanol or lactic acid.

As used herein “total fermentable sugars” or “TFS” refers to fructose, sucrose, glucose, maltose and maltotriose. Thus, the amount of the TFS is the total amount of fructose, sucrose, glucose, maltose and maltotriose.

The term “limit dextrinase” as used herein describes a sugar hydrolase belonging to the enzyme class EC 3.2.1.142. The enzyme is a starch debranching enzyme which catalyses the hydrolysis of 1,6-alpha-D-glucosidic linkages in alpha- and beta-limit dextrins of amylopectin and glycogen, in amylopectin and pullulan. In a mashing processes the availability of free limit dextranase is expected to affect the release of fermentable sugars from the starch, especially if the starch has a high degree of branching (see for example Calum et al. 2004 J Inst Brewing 110(4): 284-296). In particular, limit dextrinase may be a polypeptide of the sequence available under UniProt accession No. Q9FYY0 or a functional homologue thereof sharing at least 90%, such as at least 95% sequence identity therewith.

The term “limit dextrinase inhibitor” or “LDI” as used herein describes a polypeptide that binds to and prevents the enzymic action of the starch debranching enzyme limit dextrinase, see for example Y Stahl et al. 2007 Plant Science 172(3): 452-561.

The term “free limit dextrinase activity” or “free LD activity” when used herein means limit dextrinase which is not bound by a limit dextrinase inhibitor. When the limit dextrinase and the limit dextrinase inhibitors are bound together in a complex, limit dextrinase cannot exert its enzymatic effect. Whereas limit dextrinase not bound to a limit dextrinase inhibitor is free and can exert its enzymatic activity. If not otherwise specified the term “limit dextrinase activity” refers to “free limit dextrinase activity”.

The term “total limit dextrinase” when used herein represents both free limit dextrinases which are not bound by limit dextrinase inhibitors and inactivated limit dextrinases which are bound by limit dextrinase inhibitors. Thus, total limit dextrinase refers to both bound and unbound forms of limit dextrinases.

The term “gelatinisation temperature” as used herein refers to the peak temperature of a temperature range during which starch loses its semi-crystalline structure in water under the impact of heat and forms a gel. Preferably, gelatinisation temperature is determined as described in Example 4 below. Reference to a cereal having a particular gelatinisation temperature refers to a cereal comprising starch with said gelatinisation temperature.

The term “germinated grain” as used herein refers to a grain having developed a visible chit.

The term “initiation of germination” as used herein refers to the time point at which barley grains with a water content of less than 15% is contacted with sufficient water to initiate germination.

The term “grain” is defined to comprise the cereal caryopsis, also denoted internal seed. In addition, the kernel may comprise the lemma and palea. In most barley varieties, the lemma and palea adhere to the caryopsis and are a part of the kernel following threshing. However, naked barley varieties also occur. In these, the caryopsis is free of the lemma and palea and threshes out free as in wheat. The terms “grain” and “kernel” are used interchangeably herein.

The term “malting” as used herein refers to a controlled germination of cereal grains (in particular barley grains) taking place under controlled environmental conditions. In some embodiments “malting” may further comprise a step of drying said germinated cereal grains, e.g. by kiln drying. The malting process induces hydrolytic enzyme activity of for example alpha-amylases and limit dextrinase.

The term “malt” as used herein refers to cereal grains, which have been malted.

“Mashing” is the incubation of milled malt (e.g. green malt or kiln dried malt), and/or ungerminated cereal grains in water. Mashing is preferably performed at specific temperature(s), and in a specific volume of water. The process allows extraction of sugars, oligo- and polysaccharides, proteins and other compounds of malt and/or grains and allows enzymatic hydrolysis of oligo- and polysaccharides (notably starch) in the extract into fermentable sugars.

The term “missense mutation” as used herein refers to a mutation/mutations in an nucleotide sequence resulting in a change from one amino acid to another in the polypeptide encoded by said nucleotide sequence.

“Mutations” include deletions, insertions, substitutions, transversions, and point mutations in the coding and/or noncoding regions of a gene. Deletions may be of an entire gene, or of only a portion of a gene. Point mutations may concern changes of one base pair, and may result in premature stop codons, frameshift mutations, mutation of a splice site or amino acid substitutions. A gene comprising a mutation when compared to a wild type gene may be referred to as a “mutant gene”. In the present invention a mutant gene generally encodes a polypeptide with a sequence different to the wild type gene, said polypeptide may be referred to as a “mutant polypeptide”. A mutant polypeptide may comprise an amino acid substitution, such a substitution can for example be described as “amino acid XXX at position n has been substituted to amino acid YYY” where XXX describes the amino acid at the specific position (n) of the wild type polypeptide and YYY describes the amino acid present in the mutant polypeptide at the same position when the two genes are aligned.

The term “non-polar amino acid” as used herein refers to amino acids with a hydrophobic side chains. Preferably, the non-polar amino acid is selected from the group consisting of Alanine, Valine, Isoleucine, Leucine, Methionine, Phenylalanine, Tyrosine and Tryptophan.

The term “charged amino acid” as used herein refers to amino acids with electrically charged side chains. Preferably, the charged amino acid is selected from the group consisting of Arginine, Histidine, Lysine, Aspartic acid and Glutamic acid. Negatively charged amino acids are preferably selected from the group consisting of Aspartic acid and Glutamic acid. Positively charged amino acids are preferably selected from the group consisting of Arginine, Histidine and Lysine.

The term “non-negatively charged amino acid” as used herein refers to amino acids with side chains, which are not negatively charged. Preferably, the non-negatively charged amino acid is selected from the group consisting of Alanine, Valine, Isoleucine, Leucine, Methionine, Phenylalanine, Tyrosine, Tryptophan, Arginine, Histidine, Lysine, Serine, Threonine, Asparagine, Glutamine, Cysteine, Selenocysteine, Glycine and Proline.

The term “polar amino acid” as used herein refers to amino acids with polar, uncharged side chains. Preferably, the polar amino acid is selected from the group consisting of Serine, Threonine, Asparagine and Glutamine.

By the term “plant product” is meant a product resulting from the processing of a plant or plant material. Said plant product may thus, for example, be green malt, kiln dried malt, wort, a fermented or non-fermented beverage, a food, or a feed product.

The term “progeny” as used herein refers to any plant, which has a given plants as one of its ancestors. Progeny not only comprises direct progeny of a given plant, but also progeny after a multitude of generations, for example progeny after up to 100 generations. It may be determined whether a plant is progeny of a given parent plant by determining whether said plant carries the same mutation(s) in the HvLDI gene as said parent plant. In addition to the mutation(s) in the HvLDI gene, the presence of additional polymorphism in genes positioned in the vicinity of the HvLDI gene may be used to determine whether a plant is progeny of a given parent plant. The presence of the same polymorphisms demonstrates that the plant is progeny of said parent plant.

The term “loop regions” as used herein refers to one or more sequential groups of amino acids corresponding to amino acids from position 25 to 44 of SEQ ID NO:1, corresponding to amino acids from position 56 to 62 of SEQ ID NO:1, corresponding to amino acids from position 77 to 78 of SEQ ID NO:1, corresponding to amino acids from position 91 to 111 of SEQ ID NO:1 and/or corresponding to amino acids from position 124 to 147 of SEQ ID NO:1. The loop regions may form a loop structure, which connect the alpha helix regions described herein below.

The term “alpha helix regions” as used herein refers to one or more sequential groups of amino acids corresponding to amino acids from position 45 to 55 of SEQ ID NO:1 and corresponding to amino acids from position 63 to 76 of SEQ ID NO:1 and corresponding to amino acids from position 79 to 90 of SEQ ID NO:1 and/or corresponding to amino acids from position 112 to 123 of SEQ ID NO:1. These alpha helix regions may form a helical structure.

The term “sequence identity” as used herein describes the relatedness between two amino acid sequences or between two nucleotide sequences, i.e. a candidate sequence (e.g. a mutant sequence) and a reference sequence (such as a wild type sequence) based on their pairwise alignment. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000,Trends Genet. 16: 276-277,), preferably version 5.0.0 or later (available at https://www.ebi.ac.uk/Tools/psa/emboss_needle/). The parameters used are gap open penalty of 10, gap extension penalty of 0.5, and the EBLOSUM62 (EMBOSS version of 30 BLOSUM62) substitution matrix. The output of Needle labeled “longest identity” (obtained using the -nobrief option) is used as the percent identity and is calculated as follows:

(Identical Residues×100)/(Length of Alignment−Total Number of Gaps in Alignment)

The Needleman-Wunsch algorithm is also used to determine whether a given amino acid in a sequence other than the reference sequence (e.g. a natural variant or halotype of SEQ ID NO: 1) corresponds to a given position of SEQ ID NO: 1 (reference sequence). For example, if the natural variant has two additional amino acids in the N-terminal, position 70 in the natural variant will correspond to position 68 of SEQ ID NO: 1.

For purposes of the present invention, the sequence identity between two nucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the DNAFULL (EMBOSS version of NCBI NUC4.4) substitution matrix. The output of Needle labeled “longest identity” (obtained using the -nobrief option) is used as the percent identity and is calculated as follows:

(Identical Deoxyribonucleotides×100)/(Length of Alignment−Total Number of Gaps in Alignment).

The term “starch” as used herein refers to a composition of one or both of the discrete macromolecules: amylose and amylopectin.

The term “stop codon” as used herein refers to a nucleotide triplet in the genetic code, which within mRNA results in termination of translation. The term “stop codon” as used herein also refers to a nucleotide triplet within a gene encoding the stop codon in mRNA. The stop codon in DNA typically has one of the following sequences: TAG, TAA or TGA.

The term “wild type HvLDI” or “wt HvLDI” as used herein refers wild type barley limit dextrinase inhibitor gene or a polypeptide encoded by said gene. In nature several haplotypes of HvLDI exist, which can be considered wild type LDI. These can also be described as natural variants of the HvLDI reference sequence of SEQ ID NO:1. Thus, the term “wild type HvLDI” covers a group of wt HvLDI including the ones described by Huang et al. 2014.

By the term “wort” is meant a liquid extract of malt and/or cereal grains, such as milled malt and/or milled cereal grains and optionally additional adjuncts. Wort is in general obtained by mashing, optionally followed by “sparging”, in a process of extracting residual sugars and other compounds from spent grains after mashing with hot water. Sparging is typically conducted in a lauter tun, a mash filter, or another apparatus to allow separation of the extracted water from spent grains. The wort obtained after mashing is generally referred to as “first wort”, while the wort obtained after sparging is generally referred to as the “second wort”. If not specified, the term wort may be first wort, second wort, or a combination of both. During conventional beer production, wort is boiled together with hops. Wort without hops, may also be referred to as “sweet wort”, whereas wort boiled with hops may be referred to as “boiled wort” or simply as wort.

The term “thousand grain weight” as used herein refers to the total weight of thousand (1000) grains.

### Limit Dextrinase Inhibitor (LDI)

The present invention provides a barley plant, or a part thereof, wherein said barley plant carries a mutation in the HvLDI gene of the invention, wherein said mutated HvLDI gene encodes a mutant HvLDI polypeptide.

The coding nucleotide sequence of a wild type Hordeum vulgare limit dextrinase inhibitor (HvLDI) is available under accession no. DQ285564.1. The coding nucleotide sequence for HvLDI is also provided herein as SEQ ID NO:2. The skilled person will understand that other wild type barley plants comprise an HvLDI gene with a sequence differing from SEQ ID NO:2. These can also be described as natural variants of the HvLDI reference sequence of SEQ ID NO: 2.

The HvLDI polypeptide is available under UniProt accession No. Q2V8X0. A wt HvLDI polypetide in the context of the present invention is a polypeptide having the sequence SEQ ID NO:1 or a sequence sharing at least 90%, such as at least 93%, such as at least 95%, such as at least 98% sequence identity with SEQ ID NO:1, and wherein said sequence at least comprises the amino acids corresponding to positions 60 and 68 of SEQ ID NO:1, namely proline and glutamic acid, respectively. In other words, the amino acids corresponding to amino acids 60 and 68 of SEQ ID NO:1 are conserved in wt HvLDI.

The definition of the position of the amino acid in relation to a polypeptide of the invention is made to SEQ ID NO:1 as reference sequence, but it is understood that the sequence of the polypeptide of the invention may differ to some extend from the polypeptide sequence of SEQ ID NO: 1 (see for example the definition of wt HvLDI). Thus, is it to be understood that following alignment between said polypeptide and the reference polypeptide of SEQ ID NO:1, an amino acid corresponds to position X of SEQ ID NO:1 if it aligns to the same position.

Table 1A/1B illustrates natural wild type variants at given positions of SEQ ID NO: 1. For example, position 108 of SEQ ID NO:1 is an Arg and Wt Halotype 2 has a Thr in the position corresponding to position 108.

TABLE 1A
Polypeptides of SEQ ID: 1 (amino
acids 1 to 137 of SEQ ID NO: 1)

| SEQ ID NO: 1 | | | | | | | | | |
|---|---|---|---|---|---|---|---|---|---|
| position | 5 | 9 | 25 | 48 | 55 | 70 | 108 | 118 | 129 |
| SEQ ID NO: 1 | His | Val | Thr | His | Val | Cys | Arg | Ala | Cys |
| amino acid | | | | | | | | | |
| Wt haplotype 1 | Arg | Leu | Ala | — | — | — | — | Thr | — |
| Wt haplotype 2 | Arg | Leu | Ala | — | — | — | Thr | Thr | — |
| Wt haplotype 3 | Arg | Leu | Ala | Arg | — | — | — | Thr | — |
| Wt haplotype 4 | Arg | Leu | Ala | — | — | — | — | Thr | Phe |
| Wt haplotype 5 | Arg | Leu | Ala | — | — | — | — | Thr | — |
| Wt haplotype 6 | Arg | Leu | Ala | — | — | — | Thr | Thr | — |
| Wt haplotype 7 | Arg | Leu | — | — | — | — | — | — | — |
| Wt haplotype 8 | Arg | Leu | Ala | Arg | — | — | — | Thr | — |
| Wt haplotype 9 | Arg | Leu | Ala | — | — | Arg | — | — | — |
| Wt haplotype 10 | — | — | — | — | — | — | — | — | — |
| Wt haplotype 11 | — | — | — | — | — | — | — | — | — |
| Wt haplotype 12 | — | — | — | — | — | — | — | — | — |
| Wt haplotype 13 | — | — | — | — | — | — | — | — | — |
| Wt haplotype 14 | Arg | Leu | Ala | — | Phe | — | — | Thr | — |
| Wt haplotype 15 | — | — | — | — | — | — | — | — | — |

—: corresponds to the same amino acid as listed in the position for SEQ ID NO: 1

TABLE 1B
Polypeptides of SEQ ID: 1 (amino
acids 138 to 147 of SEQ ID NO: 1)

| SEQ ID | | | | | | | | | | |
|---|---|---|---|---|---|---|---|---|---|---|
| NO: 1 | | | | | | | | | | |
| position | 138 | 139 | 140 | 141 | 142 | 143 | 144 | 145 | 146 | 147 |
| SEQ ID | Gly | Val | Arg | Leu | Val | Leu | Leu | Ala | Asp | Gly |
| NO: 1 | | | | | | | | | | |
| amino acid | | | | | | | | | | |
| Wt | — | — | — | — | — | % | % | % | % | % |
| haplotype | | | | | | | | | | |
| 1 | | | | | | | | | | |
| Wt | — | — | — | — | — | — | — | — | — | — |
| haplotype | | | | | | | | | | |
| 2 | | | | | | | | | | |
| Wt | — | — | — | — | — | % | % | % | % | % |
| haplotype | | | | | | | | | | |
| 3 | | | | | | | | | | |
| Wt | — | — | — | — | — | — | — | — | — | — |
| haplotype | | | | | | | | | | |
| 4 | | | | | | | | | | |
| Wt | — | — | — | — | — | — | — | — | — | — |
| haplotype | | | | | | | | | | |
| 5 | | | | | | | | | | |
| Wt | — | — | — | Val | — | Ser | Pro | Asp | — | — |
| haplotype | | | | | | | | | | |
| 6 | | | | | | | | | | |
| Wt | — | — | — | — | — | — | — | — | — | — |
| haplotype | | | | | | | | | | |
| 7 | | | | | | | | | | |
| Wt | — | — | — | Trp | — | — | — | — | — | — |
| haplotype | | | | | | | | | | |
| 8 | | | | | | | | | | |
| Wt | — | — | — | — | — | — | — | — | He | — |
| haplotype | | | | | | | | | | |
| 9 | | | | | | | | | | |
| Wt | — | — | — | — | — | — | — | Thr | — | — |
| haplotype | | | | | | | | | | |
| 10 | | | | | | | | | | |
| Wt | — | — | — | — | — | % | % | % | % | % |
| haplotype | | | | | | | | | | |
| 11 | | | | | | | | | | |
| Wt | — | — | — | — | — | — | — | — | — | — |
| haplotype | | | | | | | | | | |
| 12 | | | | | | | | | | |
| Wt | — | — | — | Val | Ala | — | — | — | Met | Val |
| haplotype | | | | | | | | | | |
| 13 | | | | | | | | | | |
| Wt | Val | Leu | Leu | — | — | — | Val | — | — | — |
| haplotype | | | | | | | | | | |
| 14 | | | | | | | | | | |
| Wt | Val | Leu | Leu | — | — | — | Val | — | Met | Val |
| haplotype | | | | | | | | | | |
| 15 | | | | | | | | | | |

—: corresponds to the amino acid of the listed position of SEQ ID NO: 1
%: lacking the amino acid

Polypeptides of SEQ ID NO:1 with the substitutions mentioned in Table 1A and 1B herein above are in the context of the present invention all considered to be wt HvLDI polypeptides.

In one embodiment of the invention, the mutant HvLDI polypeptide of the invention is at least 90% identical such as 95% identical, such as 98%, such as 100% identical to SEQ ID NO:1 except for the amino acids in position 60 and/or 68.

The proposed structure of LDI is described in Stahl et al. 2004 and Møller et al. 2015. LDI is considered to consist of a signal peptide and four alpha helix regions, where the alpha helix regions are joined by loop regions. The loop regions may be selected from the group consisting of amino acids corresponding to position 25 to 44 and amino acids corresponding to position 56 to 62 and amino acids corresponding to position 77 to 78 and amino acids corresponding to position 91 to 111 and amino acids corresponding to position 124 to 147 of SEQ ID NO:1. The alpha helix regions may be selected from the group consisting of amino acids corresponding to position 45 to 55 and amino acids corresponding to position 63 to 76 and amino acids corresponding to position 79 to 90 and amino acids corresponding to position 112 to 123 of SEQ ID NO:1. The signal peptide corresponds to amino acids from 1 to 24 of SEQ ID NO:1.

Furthermore, mature HvLDI polypeptides, without the signal peptide is also considered wt HvLDIs, including all the polypeptides in Table 1 A and 1B without the amino acids corresponding to position 1 to 24 of SEQ ID NO: 1. In particular, the mature polypeptide of SEQ ID NO:1 without amino acids at position 1 to 24 of SEQ ID NO:1 is considered wt HvLDI. In other words, a polypeptide consisting of amino acids from 25 to 147 of SEQ ID NO:1 is considered wt HvLDI.

The amino acids of the mature HvLDI polypeptide, without the signal peptide, can be described in relation to amino acids of HvLDI of SEQ ID NO:1. Thus, the amino acid positions of the mature HvLDI polypeptide can be calculated based on the amino acid position of SEQ ID NO:1 by subtracting 24 amino acids from the amino acid position of SEQ ID NO:1. This is for example the case with the amino acid positions used in Møller et al. 2015.

One example hereof is that the amino acid at position 60 of the HvLDI polypeptide of SEQ ID NO:1 corresponds to amino acid at position 36 of the mature HvLDI polypeptide. Another example hereof is that the amino acid at position 66 of the HvLDI polypeptide of SEQ ID NO:1 corresponds to amino acid at position 42 of the mature HvLDI polypeptide. Another example hereof is that the amino acid at position 68 the HvLDI polypeptide of SEQ ID NO:1 corresponds to amino acid at position 44 of the mature HvLDI polypeptide.

A wt HvLDI gene is a gene encoding a wt HvLDI polypeptide. In particular, the wt HvLDI gene may be the nucleotide sequence of SEQ ID:2 or a functional homologue thereof sharing at least 90%, such as at least 93%, such as at least 95%, such as at least 98% sequence identity therewith.

Preferably, a wt HvLDI gene is a gene encoding a wt HvLDI polypeptide which shares at least 90%, such as at least 93%, such as at least 95%, such as at least 98%, sequence identity to SEQ ID NO:2 wherein said sequence at least comprises the nucleic acid at positions 966, and 990 of SEQ ID NO:2, namely a C and G respectively. More specifically, the polypeptide encoded by a wt HvLDI gene preferably comprises a proline at amino acid position 60 of SEQ ID NO:1 and a glutamic acid at amino acid position 68 of SEQ ID NO:1.

The present invention provides a barley plant, or part thereof, wherein said barley plant carries a mutation in the HvLDI gene, wherein said mutated HvLDI gene encodes a mutant HvLDI polypeptide, wherein the mutation is one of the following mutations
a. a missense mutation resulting in a change from a proline to a different amino acid in one or more loop regions of HvLDI, wherein the loop regions are selected from the group consisting of amino acids corresponding to position 25 to 44 and amino acids corresponding to position 56 to 62 and amino acids corresponding to position 77 to 78 and amino acids corresponding to position 91 to 111 and amino acids corresponding to position 124 to 147 of SEQ ID NO:1; or
b. a missense mutation resulting in a change from a negatively charged amino acid to a non-negatively charged amino acid in one or more alpha helix regions of wt HvLDI, wherein the alpha helix regions are selected from the group consisting of amino acids corresponding to position 45 to 55 and amino acids corresponding to position 63 to 76 and amino acids corresponding to position 79 to 90 and amino acids corresponding to position 112 to 123 of SEQ ID NO:1.

In one embodiment, said mutant HvLDI polypeptide is identical to the mature wt HvLDI polypeptide or natural variants thereof apart from the mutation in the specified position(s).

The present invention also provides a barley plant or part thereof, wherein said barley plant carries one or more mutations in the HvLDI gene selected from the group consisting of:
a. a mutation of nucleotide C to T at the position corresponding to nucleotide 966 of the coding sequence of the HvLDI gene (SEQ ID NO:2); and
b. a mutation of nucleotide C to T at the position corresponding to nucleotide 967 of the coding sequence of the HvLDI gene (SEQ ID NO:2); and
c. a mutation of nucleotide C to T at the position corresponding to nucleotide 968 of the coding sequence of the HvLDI gene (SEQ ID NO:2); and
d. a mutation of G to A at the position corresponding to nucleotide 990 of the coding sequence of the HvLDI gene (SEQ ID NO:2)

### Missense Mutation Resulting in a Change from a Proline to a Different Amino Acid

In some embodiments of the invention, the mutant HvLDI polypeptide comprises a substitution of a proline to a different amino acid in one or more of the loop regions of HvLDI. The substitution of a proline may be to a polar amino acid or a non-polar amino acid. In particular, it may be a substitution of a proline to a serine or leucine. It is preferred that the substitution of the proline is at amino acid corresponding to position 60 of SEQ ID NO:1.

In some embodiments of the present invention, said mutated HvLDI gene encodes a mutant HvLDI polypeptide, wherein said mutant HvLDI polypeptide comprises a substitution of a proline to a different amino acid in one or more of the loop regions of HvLDI, wherein the loop regions are selected from the group consisting of amino acids corresponding to position 25 to 44 and amino acids corresponding to position 56 to 62 and amino acids corresponding to position 77 to 78 and amino acids corresponding to position 91 to 111 and amino acids corresponding to position 124 to 147 of SEQ ID NO:1.

In one embodiment, said mutated HvLDI gene encodes a mutant HvLDI polypeptide, wherein said mutated HvLDI gene encodes a mutant HvLDI polypeptide, wherein the loop regions are selected from the group consisting of amino acids corresponding to position 56 to 62 and amino acids corresponding to position 77 to 78 and amino acids corresponding to position 91 to 111 of SEQ ID NO:1.

In one embodiment, said mutated HvLDI gene encodes a mutant HvLDI polypeptide, wherein said mutant HvLDI polypeptide comprises a substitution of a proline in one or more of the loop regions of wt HvLDI to a polar amino acid.

In one embodiment, said mutated HvLDI gene encodes a mutant HvLDI polypeptide, wherein said mutant HvLDI polypeptide comprises a substitution of a proline at amino acid corresponding to position 60 of SEQ ID NO:1 to a different amino acid.

In another embodiment, said mutated HvLDI gene encodes a mutant HvLDI polypeptide, wherein said HvLDI polypeptide comprises a substitution of proline to serine in one or more of the loop regions of HvLDI.

In one embodiment, it is preferred that said mutated HvLDI gene encodes a mutant HvLDI polypeptide, wherein said mutant HvLDI polypeptide comprises a substitution of a proline at amino acid corresponding to position 60 of SEQ ID NO:1 to serine.

In one embodiment, the mutant HvLDI polypeptide of the present invention comprises or consists of the amino acid sequence from position 25 to 142 of SEQ ID NO: 3 or from position 25 to 147 of SEQ ID NO: 3.

In some embodiments of the present invention, said mutated HvLDI gene encodes a mutant HvLDI, wherein said mutant HvLDI polypeptide comprises a substitution of a proline in one or more of the loop regions of wt HvLDI to a non-polar amino acid.

In one embodiment, said mutated HvLDI gene encodes a mutant HvLDI polypeptide, wherein said mutant HvLDI polypeptide comprises a substitution of a proline in one or more of the loop regions of HvLDI to a leucine.

In another embodiment, it is preferred that said mutated HvLDI gene encodes a mutant HvLDI polypeptide, wherein said mutant HvLDI polypeptide comprises a substitution of a proline at amino acid corresponding to position 60 of SEQ ID NO:1 to leucine.

In one embodiment, the mutant HvLDI polypeptide of the present invention comprises or consists of the amino acid sequence from position 25 to 142 of SEQ ID NO: 4 or from position 25 to 147 of SEQ ID NO: 4

In one embodiment said mutated HvLDI gene contain a mutation of nucleotide C to T at the position corresponding to nucleotide 966 of the HvLDI reference gene of SEQ ID NO:2.

In another embodiment said mutated HvLDI gene contain a mutation of nucleotide C to T at the position corresponding to nucleotide 967 of the HvLDI reference gene of SEQ ID NO:2.

In one embodiment said mutated HvLDI gene contain a mutation of nucleotide C to T at the position corresponding to nucleotide 966 and 967 of the HvLDI reference gene of SEQ ID NO:2.

In one embodiment said mutated HvLDI gene contain a mutation of nucleotide C to T at the position corresponding to nucleotide 966 and/or 968 of the HvLDI reference gene of SEQ ID

In one embodiment the barley plant of the invention comprises a mutant HvLDI gene encoding a mutant HvLDI protein having a Pro60Ser mutation of SEQ ID NO: 1. For example, the barley plant may comprise a mutant HvLDI gene carrying a C→T mutation of the nucleotide 966 of the HvLDI coding sequence of SEQ ID NO:2. Said barley plant may for example be HENZ-16a or progeny thereof. HENZ-16a may also be referred to as “HENZ-16” herein. For example, the barley plant may be a HENZ-16a barley plant identified as described in Example 1 or progeny thereof.

For the purposes of this patent application seeds of barley plant (Hordeum vulgare) designated “HENZ-16” (also referred to as “HENZ-16a” herein) has been deposited with NCIMB Ltd. Ferguson Building, Craibstone Estate, Bucksburn, Aberdeen, AB21 9YA Scotland under the provisions of the Budapest Treaty. The HENZ-16 barley plant was deposited on 12 Feb. 2020 and has received the accession number NCIMB 43581.

In one embodiment, the barley plant of the invention is the barley plant (Hordeum vulgare) deposited on 12 Feb. 2020 with NCIMB under the accession number NCIMB 43581 and referred to as “HENZ-16” or progeny thereof. Thus, the barley plant of the invention may be barley plant HENZ-16 deposited with NCIMB on 12 February 2020 and having accession number NCIMB 43581 or any progeny barley plant thereof, wherein the progeny barley plant carries a C→T mutation of nucleotide 966 of the HvLDI coding sequence of SEQ ID NO:2 and/or wherein the HvLDI gene of said barley plant encodes a mutant HvLDI protein comprising a Pro60Ser mutation of SEQ ID NO: 1.

Missense Mutation Resulting in a Change from a Negatively Charged Amino Acid to a Non-Negatively Charged Amino Acid

In some embodiments of the invention, the mutant HvLDI polypeptide comprises a substitution of a negatively charged amino acid to a non-negatively charged amino acid in one or more of the alpha helix regions of HvLDI. The substitution of the negatively charged amino acid may be to a positively charged amino acid. In particular, it may be a substitution of a negatively charged amino acid to a lysine. It is preferred that the substitution of the negatively charged amino acid is the amino acid corresponding to position 68 of SEQ ID NO:1.

In some embodiments of the present invention, said mutated HvLDI gene encodes a mutant HvLDI polypeptide, wherein said mutant HvLDI polypeptide comprises a substitution of a negatively charged amino acid in one or more of the alpha helix regions of HvLDI to a non-negatively charged amino acid. In one embodiment, said negatively charged amino acid is glutamic acid.

In some embodiments of the present invention, said mutated HvLDI gene encodes a mutant HvLDI polypeptide, wherein said mutant HvLDI polypeptide comprises a substitution of a negatively charged amino acid in one or more of the alpha helix regions of HvLDI to a positively charged amino acid. In one embodiment, said negatively charged amino acid is glutamic acid.

In one embodiment, said mutated HvLDI gene encodes a mutant HvLDI polypeptide, wherein said mutant HvLDI polypeptide comprises a substitution of a negatively charged amino acid in one or more of the alpha helix regions of HvLDI to a lysine. In one embodiment, said negatively charged amino acid is glutamic acid.

In some embodiments the alpha helix regions are selected from the group consisting of amino acids corresponding to position 45 to 55 and amino acids corresponding to position 63 to 76 and amino acids corresponding to position 79 to 90 and amino acids corresponding to position 112 to 123 of SEQ ID NO:1

In another embodiment, said mutated HvLDI gene encodes a mutant HvLDI polypeptide, wherein said mutant HvLDI polypeptide comprises a substitution of a glutamic acid corresponding to the amino acid at position 68 of SEQ ID NO:1 to a non-negatively charged amino acid.

In yet another embodiment, said mutated HvLDI gene encodes a mutant HvLDI polypeptide, wherein said mutant HvLDI polypeptide comprises a substitution of a glutamic acid corresponding to the amino acid position 68 of SEQ ID NO:1 to a lysine.

In one embodiment, the mutant HvLDI polypeptide of the present invention comprises or consists of the amino acid sequence from position 25 to 142 of SEQ ID NO: 6 or from position 25 to 147 of SEQ ID NO: 6.

In one embodiment said mutated HvLDI gene contain a mutation of nucleotide G to A at the position corresponding to nucleotide 990 of the HvLDI reference gene of SEQ ID NO:2.

In one embodiment said mutated HvLDI gene contain a mutation of nucleotide C to T at the position corresponding to nucleotide 966 and of nucleotide G to A at the position corresponding to nucleotide 990 of the HvLDI reference gene of SEQ ID NO:2.

In one embodiment said mutated HvLDI gene contain a mutation of nucleotide C to T at the position corresponding to nucleotide 967 and of nucleotide G to A at the position corresponding to nucleotide 990 of the HvLDI reference gene of SEQ ID NO:2

In one embodiment the barley plant of the invention comprises a mutant HvLDI gene encoding a mutant HvLDI protein having a Glu68Lys mutation of SEQ ID NO: 1. For example, the barley plant may comprise a mutant HvLDI gene carrying a G—A mutation of the nucleotide 990 of the HvLDI coding sequence of SEQ ID NO:2. Said barley plant may for example be HENZ-31 or progeny thereof. For example, the barley plant may be a HENZ-31 barley plant identified as described in Example 1 or progeny thereof.

For the purposes of this patent application seeds of barley plant (Hordeum vulgare) designated “HENZ-31” have been deposited with NCIMB Ltd. Ferguson Building, Craibstone Estate, Bucksburn, Aberdeen, AB21 9YA Scotland under the provisions of the Budapest Treaty. The HENZ-31 barley plant was deposited on 12 February 2020 and has received the accession number NCIMB 43582.

In one embodiment, the barley plant of the invention is the barley plant (Hordeum vulgare) deposited on 12 February 2020 with NCIMB under the accession number NCIMB 43582 and referred to as “HENZ-31”; or progeny thereof. Thus, the barley plant of the invention may be barley plant HENZ-31 deposited with NCIMB on 12 Feb. 2020 and having accession number NCIMB 43582 or any progeny barley plant thereof, wherein the progeny barley plant carries a G→A mutation of nucleotide 990 of the HvLDI coding sequence of SEQ ID NO:2 and/or wherein the HvLDI gene of said barley plant encodes a mutant HvLDI protein comprising a Glu68Lys mutation of SEQ ID NO: 1.

### HvLDI Activity

As mentioned herein above, HvLDI is able to bind to HvLD and hereby inhibits the activity of HvLD. Active HvLD is able to cleave alpha-1-6 linkages in branched dextrins molecules.

The ability of HvLDI to inhibit HvLD in vitro can be measured by any useful method known to the skilled person. For such methods, recombinant HvLD and HvLDI can be manufactured according to known methods or be purchased from standard suppliers. Commercially available assays for assessing the binding affinity between HvLD and HvLDI, is for example the Pullulanase/Limit-Dextrinase Assay Kit from Megazyme, Ireland.

A preferred in vitro method for determining the binding affinity between HvLDI and HvLD is described in Example 2.

A mutant HvLDI polypeptide carrying a mutation according to the present invention have a reduced ability to inhibit HvLD when assessed according to the assay of Example 2.

A mutant HvLDI polypeptide carrying a mutation according to the present invention preferably has at least a 2-fold decreased ability to inhibit HvLD, such as at least 3-fold decreased ability to inhibit HvLD compared to ability of the wt HvLDI to inhibit HvLD when measured in vitro. In one embodiment of the present invention, the mutated HvLDI is not able to fully inhibit the HvLD activity when measured in vitro.

In one embodiment of the present invention, the mutation in the HvLDI gene according to the present invention results in an increased free HvLD activity, when compared to a wild type HvLDI gene.

### Barley Plant

The present invention relates to a barley plant, or part thereof, as well as barley products and method of producing these. The barley plant may be any plant of the species Hordeum vulgare, including any breeding line or cultivar or variety.

“Wild barley”, Hordeum vulgare ssp. spontaneum, is considered the progenitor of today's cultivated forms of barley. Domesticated, but heterogenous mixtures of barley are referred to as barley landraces. Today, most of the landraces have been displaced in advanced agricultures by pure line cultivars. Compared with landraces, modern barley cultivars have numerous improved properties (Nevo, 1992; Pelger et al., 1992).

Within the present invention, the term “barley plant” comprises any barley plant, such as barley landraces or modern barley cultivars. Thus, the invention relates to any barley plant comprising a mutation in the HvLDI gene of the invention.

However, preferred barley plants for use with the present invention are modern barley cultivars or pure lines. Non-limiting examples of barley cultivars, which can be used with the present invention include Planet, Paustian, Sebastian, Quench, Celeste, Lux, Prestige, Saloon, Neruda, Harrington, Klages, Manley, Schooner, Stirling, Clipper, Franklin, Alexis, Blenheim, Ariel, Lenka, Maresi, Steffi, Gimpel, Cheri, Krona, Camargue, Chariot, Derkado, Prisma, Union, Beka, Kym, Asahi 5, KOU A, Swan Hals, Kanto Nakate Gold, Hakata No. 2, Kirin — choku No. 1, Kanto late Variety Gold, Fuji Nijo, New Golden, Satukio Nijo, Seijo No. 17, Akagi Nijo, Azuma Golden, Amagi Nijpo, Nishino Gold, Misato golden, Haruna Nijo, Scarlett, Rosalina and Jersey preferably from the group consisting of Haruna Nijo, Sebastian, Quench, Celeste, Lux, Prestige, Saloon, Neruda and Power, preferably from the group consisting of Paustian, Harrington, Klages, Manley, Schooner, Stirling, Clipper, Franklin, Alexis, Blenheim, Ariel, Lenka, Maresi, Steffi, Gimpel, Cheri, Krona, Camargue, Chariot, Derkado, Prisma, Union, Beka, Kym, Asahi 5, KOU A, Swan Hals, Kanto Nakate Gold, Hakata No. 2, Kirin—choku No. 1, Kanto late Variety Gold, Fuji Nijo, New Golden, Satukio Nijo, Seijo No. 17, Akagi Nijo, Azuma Golden, Amagi Nijpo, Nishino Gold, Misato golden, Haruna Nijo, Scarlett and Jersey preferably from the group consisting of Planet, Paustian, Haruna Nijo, Sebastian, Tangent, Lux, Prestige, Saloon, Neruda, Power, Quench, NFC Tipple, Barke, Class, Vintage, Applaus, Bowie, Broadway, Champ, Chanson, Charles, Chimbon, Cosmopolitan, Crossway, Dragoon, Ellinor, Embrace, Etoile, Evergreen, Flair, Highway, KWS Beckie, KWS Cantton, KWS Coralie, KWS Fantex, KWS Irina, KWS Josie, KWS Kellie, LG Diablo, LG Figaro, LG Nabuco, LG Tomahawk, Laureate, Laurikka, Lauxana, Luther, Odyssey, Ovation, Prospect, RGT Elysium, RGT Observer, RGT Planet, Rotator, Sarbi, Scholar, Subway or Golden Promise.

The barley plant may be in any suitable form. For example, the barley plant according to the invention may be a viable barley plant, a dried plant, a homogenized plant, or a milled barley kernel. The plant may be a mature plant, an embryo, a kernel, a germinated kernel, a malted kernel, a milled malted kernel, a milled kernel or the like.

Parts of barley plants may be any suitable part of the plant, such as grains, embryos, leaves, stems, roots, flowers, or fractions thereof. A fraction may, for example, be a section of a kernel, embryo, leaf, stem, root, or flower. Parts of barley plants may also be a fraction of a homogenate or a fraction of a milled barley plant or kernel.

In one embodiment of the invention, parts of barley plants may be cells of said barley plant, such as viable cells that may be propagated in vitro in tissue cultures. In other embodiments, however, the parts of barley plants may be viable cells that are not capable of maturing into an entire barley plant, i.e. cells that are not a reproductive material.

It is preferred that the barley plant has not exclusively been obtained by means of an essentially biological process or is progeny thereof. For example, the barley plant may comprise a mutation in the HvLDI gene, wherein said mutation has been induced by chemical and/or physical agents, such as sodium azide.

Thus, the barley plant may have been prepared by a method involving a step of induced mutagenesis or said barley plant may be progeny of a plant prepared by a method involving a step of induced mutagenesis. Said induced mutagenesis may for example be treatment with a mutagenizing chemical, such as sodium azide.

The barley plant may also be a barley plant prepared by genetic engineering techniques, for example by inserting the mutated HvLDI gene into the host genome using plasmids or genetic recombination or the Crisper/CAS-9 technology. Preferably, the wt HvLDI gene has been knocked out in such plants.

In some embodiments of the invention, the barley plant, or part thereof, carries a mutation in the HvLDI gene according to the invention.

In addition to the mutation in the HvLDI gene, the barley plant may comprise other mutations.

### Characteristics of Barley Plants Carrying a Mutation in the HvLDI Gene

The present invention provides barley plants, or parts thereof, carrying a mutation in the HvLDI gene of the invention, said mutated HvLDI gene encodes a mutant HvLDI polypeptide. One major advantage of such barley plants is that the grains of said barley plants according to the invention have an increased free HvLD activity compared to barley plants with wt HvLDI gene. Furthermore, malt prepared from such barley grains also have a higher level of free HvLD activity. Surprisingly, the increased free HvLD activity correlates well with the concentration of fermentable sugars in wort prepared from the grains of said barley plants, whereas wort prepared from barley grains with low or normal levels of free HvLD activity in general have low or normal concentration of fermentable sugars. Thus, increased free HvLD activity is advantageous trait for barley wort and beer production.

The amount of free HvLD activity in barley can be measured by any useful method known to the skilled person. Typically, the first step is crushing one or more barley grains, e.g. by mechanical means to obtain barley flour. This may be done by any useful means, e.g. by hydraulic press and/or by use of a grinder and/or a mill, but the exact method of mechanical crushing is of less importance.

One non-limiting example of a useful method for measuring free HvLD activity may be to extract the obtained flour into an acidic aqueous solution, such as maleic acid, at pH 4.7 for 1 h at 40° C. The free HvLD activity may be analyzed by the PullG6 method from Megazyme.

A preferred method of determining the free HvLD activity in barley is described in Example 7.

In some embodiments of the present invention, said barley plant, or part thereof, carrying a mutation in the HvLDI gene of the invention, have a higher free HvLD activity compared to a barley plant, or part thereof, carrying a HvLDI gene encoding a wt HvLDI, but otherwise of the same genotype, when cultivated under the same conditions.

In one embodiment of the present invention, the barley plant or parts thereof or germinated grains or malt prepared from grains of a barley plant of the invention carrying a mutation in the HvLDI gene of the invention, have a free HvLD activity at least 20% higher compared to the free HvLD activity measured in malt of barley plants carrying a HvLDI gene encoding a wt HvLDI, but otherwise of the same genotype, when prepared under the same conditions. In some embodiments, said free HvLD activity is at least 50% higher, such as at least 100% higher, such as at least 140% higher compared to the free HvLD activity measured in malt of barley plants carrying a HvLDI gene encoding a wt HvLDI, but otherwise of the same genotype, when prepared under the same conditions. In one embodiment, said gains have been germinated for 72 hours prior to the measurement.

The total HvLD activity may be determined by any useful method. Typically, such methods comprise incubation under conditions disrupting binding between LDI and LD followed by determination of LD activity. One non-limiting example of measuring total HvLD may be to extract the obtained flour into a redox reagent, such as dithiothreitol, at pH 4.7 for 1 h at 40° C. The amount of HvLD activity may then be analyzed as described above. The HvLD activity determined after incubation with the redox reagent will reflect the total HvLD activity.

A preferred method of determining the total HvLD activity is described in Example 7.

In some embodiments of the present invention, said barley plant, or part thereof, carrying a mutation in the HvLDI gene of the invention, have a higher ratio of free to total HvLD activity compared to a barley plant, or part thereof, carrying a HvLDI gene encoding a wt HvLDI, but otherwise of the same genotype, when cultivated under the same conditions. The ratio of free to total HvLD may be provided as free/total (%).

In another embodiment of the present invention, the barley plant or parts thereof or germinated grains or malt thereof, carrying a mutation in the HvLDI gene of the invention, have a free/total HvLD activity at least 20% higher compared to the free/total % HvLD activity measured in malt of barley plants carrying a HvLDI gene encoding a HvLDI of SEQ ID NO:1, but otherwise of the same genotype, when prepared under the same conditions. In some embodiments, said free/total % HvLD activity is at least 30% higher, such as at least 40% higher, such as at least 50% higher compared to the free/total % HvLD activity measured in malt of barley plants carrying a HvLDI gene encoding a wt HvLDI, but otherwise of the same genotype, when prepared under the same conditions.

In one embodiment of the invention the ratio between free limit dextrinase and total limit dextrinase is at least 35% in flex-malt malted grains and at least 60% in conventionally malted grains (e.g. kiln dried malt).

The barley plants of the present invention generally have physical appearance and grain yield comparable to barley plants which do not carry a mutation in the HvLDI gene. Thus, grains of a barley plant of the invention also generally have gelatinization temperature, alpha-amylase activity, beta-amylase activity, amylopectin chain length distribution, weight, size, protein content, water content and starch content comparable to grains of a wild type barley plant of the same genotype cultivated under the same conditions.

In one embodiment, grains of a barley plant according to the present invention have essentially the same germination ability compared to grains of a barley plant, which do not carry a mutation in the HvLDI gene of the invention. For example, in one embodiment, grains of a barley plant according to the invention have essentially the same germination index calculated through a period of 3 days using the equation 10*(x+y+z)/(x+2*y+3*z), wherein x is number of germinating grains counted at 24 hr, y is the number of germinating grains counted at 48 hr and z is the number of germinating grains counted at 72 hr compared to grains from a wt barley plant of the same genotype and prepared under similar or the same conditions. In another embodiment, grains of a barley plant according to the invention have essentially the same germination percentage at 24 hr, 48 hr and 72 hr, calculated using the number of germinated grains counted at 24 hr in relation to the total amount of grains at 24 hr, using the number of germinated grains counted at 48 hr in relation to the total amount of grains at 48 hr, and using the number of germinated grains counted at 72 hr in relation to the total amount of grains at 72 hr compared to grains from a wt barley plant of the same genotype and prepared under similar or the same conditions For example, in yet another embodiment, grains of a barley plant according to the invention have essentially the same water sensitivity as measured by counting germinated grains after 72 hours incubation with 8 ml liquid compared to germinated grains after 72 hours incubated with 4 ml liquid, compared to water sensitivity in grains from a wt barley plant of the same genotype and prepared under similar or the same conditions.

In one embodiment, grains of a barley plant according to the present invention have essentially the same gelatinization temperature (° C.) compared to grains of a barley plant, which do not carry a mutation in the HvLDI gene of the invention. For example, in one embodiment the invention provides grains of a barley plant, wherein the starch of said grains have an average gelatinisation temperature, which is similar to the average gelatinisation temperature of starch of grains of a barley plant not carrying said mutation, but otherwise identical; and grown under similar or the same conditions.

In one embodiment, grains of a barley plant according to the present invention have essentially the same alpha-amylase activity compared to grains of a barley plant, which do not carry a mutation in the HvLDI gene of the invention. In one embodiment the invention provides grains of a barley plant, wherein the alpha-amylase activity of said grains is similar to the alpha-amylase activity in grains of a barley plant not carrying said mutation, but otherwise identical; and grown under similar or the same conditions. In another embodiment the invention provides malt prepared from grains of a barley plant, wherein the alpha-amylase activity in said malt is similar to the alpha-amylase activity in malt prepared from grains of a barley plant not carrying said mutation, but otherwise identical; and grown and prepared under similar or the same conditions.

In one embodiment, grains of a barley plant according to the present invention have essentially the same beta-amylase activity compared to grains of a barley plant, which do not carry a mutation in the HvLDI gene of the invention. In one embodiment the invention provides grains of a barley plant, wherein the beta-amylase activity in said grains is similar to the beta-amylase activity in grains of a barley plant not carrying said mutation, but otherwise identical; and grown under similar or the same conditions. In another embodiment, the invention provides malt prepared from grains of a barley plant, wherein the beta-amylase activity in said grains is similar to the beta-amylase activity in malt prepared from grains of a barley plant not carrying said mutation, but otherwise identical; and grown and prepared under similar or the same conditions.

In one embodiment, grains of a barley plant according to the present invention have essentially the same amylopectin chain length distribution compared to grains of a barley plant, which do not carry a mutation in the HvLDI gene of the invention. For example, in one embodiment grains of barley plants of the invention may contain amylopectin having the same amylopectin chain length distribution compared to amylopectin of grains of a barley plant not carrying said mutation, but otherwise identical; and grown under similar or the same conditions.

In one embodiment, grains of a barley plant according to the present invention have essentially the same weight compared to grains of a barley plant, which do not carry a mutation in the HvLDI gene of the invention. In particular, grains of a barley plant of the invention may have the same average grain weight compared to the average grain weight of grains of a barley plant not carrying said mutation, but otherwise identical; and grown under similar or the same conditions.

In one embodiment, grains of a barley plant according to the invention have a thousand grain weight of at least 40 gram, such as at least 45, such as at least 50 gram, such as at least 55 gram.

In one embodiment, grains of a barley plant according to the invention have a thousand grain weight of at least 80%, such as at least 85%, such as at least 90%, such as at least 95% compared to the thousand grain weight of grains of a barley plant carrying a HvLDI gene encoding a wt HvLDI polypeptide, but otherwise of the same genotype when grown under the same conditions.

In one embodiment, a grain of a barley plant according to the invention has a weight of at least 40 gram, such as at least 45 gram, such as at least 50 gram, such as at least 55 gram.

In one embodiment, a grain of a barley plant according to the invention has a grain weight of least 80%, such as at least 85%, such as at least 90%, such as at least 95% compared to a grain from a barley plant carrying a HvLDI gene encoding a wt HvLDI polypeptide, but otherwise of the same genotype, when grown under the same conditions.

In one embodiment, grains of a barley plant according to the present invention have essentially the same size compared to grains of a barley plant, which do not carry a mutation in the HvLDI gene of the invention. Preferably, grains of a barley plant of the invention may have a grain diameter that is the same compared to the grain diameter of grains of a barley plant not carrying said mutation, but otherwise identical; and grown under similar or the same conditions.

In one embodiment, grains of a barley plant according to the present invention have essentially the same protein content compared to grains of a barley plant, which do not carry a mutation in the HvLDI gene of the invention. Preferably, grains of a barley plant of the invention may have a protein content that is the same compared to the protein content of grains of a barley plant not carrying said mutation, but otherwise identical; and grown under similar or the same conditions.

In one embodiment, grains of a barley plant according to the present invention have essentially the same water content compared to grains of a barley plant, which do not carry a mutation in the HvLDI gene of the invention. In one embodiment, grains of a barley plant of the invention may have a water content that is the same compared to the water content in grains of a barley plant not carrying said mutation, but otherwise identical; and grown under similar or the same conditions.

In one embodiment, grains of a barley plant according to the present invention have essentially the same starch content compared to grains of a barley plant, which do not carry a mutation in the HvLDI gene of the invention. For example, in one embodiment grains of barley plants of the invention may contain starch which is comparable to starch in grains of a barley plant not carrying said mutation, but otherwise identical; and grown under similar or the same conditions.

In one embodiment, grains of a barley plant according to the invention have a starch content (% of dry weight) of at least 50%, such as at least 55%, such as at least 60%.

In one embodiment, grains of a barley plant according to the invention have a starch content of at least 80%, such as at least 85%, such as at least 90%, such as at least 95% compared to grains of a barley plant carrying a HvLDI gene encoding a wt HvLDI polypeptide, but otherwise of the same genotype, when grown under the same conditions.

In one embodiment, barley plants according to the present invention have a grain yield, which is comparable to the grain yield of barley plants, which do not carry a mutation in the HvLDI gene of the invention. For example, the barley plants of the invention may in one embodiment contain a grain yield which is similar compared to the grain yield of a barley plant not carrying said mutation, but otherwise identical; and grown under similar or the same conditions.

In one embodiment, barley plants according to the invention have a grain yield of at least 80%, such as 90%, such as 95% compared to a grain yield of a barley plant carrying a HvLDI gene encoding a wt HvLDI polypeptide, but otherwise of the same genotype, when grown under the same conditions.

### Barley Plants Comprising More Than One Mutation

The present invention relates to a barley plant, or part thereof, as well as products of said barley plant and method of producing these, wherein the barley plant carry a mutation in the HvLDI gene, e.g. any of the mutations in the HvLDI gene described herein.

In addition to said mutation in the HvLDI gene, further barley plants of the invention may comprise one or more additional mutations in one or more additional genes.

In addition to the mutation in the HvLDI gene described herein, the barley plants of the invention may also comprise a mutation in the gene encoding lipoxygenase-1 (LOX-1) (SEQ ID NO: 1 in WO 2005/087934 or GenBank accession number LC099006.1) resulting in a total loss of functional LOX-1. Said mutation may for example be any of the mutations described in international patent application WO 2005/087934. For example the barley plant may comprise a gene encoding LOX-1 comprising a premature stop codon, said codon corresponding to base nos. 3572-3574 of SEQ ID NO:2 of WO 2005/087934 or a splice site mutation, said mutation corresponding to base no. 2311 of SEQ ID NO: 6 of SEQ ID NO:2 of WO 2005/087934. The loss of function of LOX-1 results in reduced amounts of free trans-2-nonenal (T2N) in a beverage produced using such a barley plant. Preferably, the T2N is at the most 0.05 ppb after incubation at 37° C. for 4 weeks, in the presence of in the range of 4 to 6 ppm sulfite.

In addition to the mutation in the HvLDI gene described herein, the barley plants of the invention may also comprise a mutation in the gene encoding lipoxygenase-2 (LOX-2) resulting in a total loss of functional LOX-2. Said mutation may for example be any of the mutations described in international patent application WO 2010/075860. For example the barley plant may comprise a gene encoding LOX-2 comprising a mutation at nucleotide position 2689 of SEQ ID NO:1 of WO 2010/075860, leading to formation of a premature stop codon. The loss of function of LOX-2 results in reduced amounts of free trans-2-nonenal (T2N) and in particular reduced amounts of T2N potential in a beverage produced using such a barley plant.

In addition to the mutation in the HvLDI gene described herein, the barley plants of the invention may also comprise a mutation in the gene encoding methionine S-methyltransferase (MMT) (SEQ ID NO: 1 in WO 2010/063288 or GenBank accession no. AB028870) resulting in a total loss of functional MMT. Said mutation may for example be any of the mutations described in international patent application WO 2010/063288. For example the barley plant may comprise a gene encoding MMT comprising a G→A mutation of base no. 3076 of SEQ ID NO:3 of WO 2010/063288 or a gene encoding MMT comprising a G→A mutation of base no. 1462 of SEQ ID NO:16 WO 2010/063288. The loss of function of MMT results in reduced amounts of dimethyl sulfide (DMS) in both green malt and kilned malt as well as beverages made from such malt. Preferably, a beverage prepared from a barley plant with MMT loss of function contains less than 30 ppm of DMS. The loss of function of MMT also results in reduced amounts of S.methyl-L-methionine (SMM) in both green malt and kilned malt as well as beverages made from such malt. Preferably, a beverage prepared from a barley plant with MMT loss of function contains less than 30 ppm of SMM.

In addition to the mutation in the HvLDI gene described herein, the barley plants of the invention may also comprise a mutation in the gene encoding cellulose synthase-like F6 (CsIF6) (SEQ ID NO: 1 in WO 2019/129736 or genbank accession number EU267181.1), wherein said mutant gene encodes mutant CsIF6 protein with reduced CsIF6 activity. Said mutation may for example be any of the mutations described in international patent application WO 2019/129736. For example the barley plant may comprise a gene encoding CsIF6 encoding mutant CsIF6 comprising a G847E mutation, or a G748D mutation or a T709I mutation of SEQ ID NO:1 or SEQ ID NO:3 of WO 2019/129736. The barley kernels with reduced CsIF6 activity have a reduced (1,3; 1,4)-beta-glucan content. High (1,3; 1,4)-beta-glucan content in the malt can form highly viscous aqueous solutions that slow filtration processes in the brewery and contribute to undesirable haze in the final beverage.

In addition to the mutation in the HvLDI gene described herein, the barley plants of the invention may also comprise any of the mutations leading to increased alpha-amylase activity described in international patent application WO 2019/129739. In particular, the barley plants of the invention may comprise a mutation in the Hordeum repressor of transcription (HvHRT) gene (SEQ ID NO: 1 in WO 2019/129739 or NCBI accession nr. AK362734.1) leading to a loss of HRT function. Said mutation in the HvHRT gene may for example be any of the mutations in the HvHRT gene described in international patent application WO 2019/129739. For example the barley plant may comprise a gene encoding HRT comprising a premature stop codon. Said mutation of the HvHRT gene may for example be a G→A mutation of nucleotide 1293 of the HvHRT coding sequence of SEQ ID NO:1 of WO 2019/129739 and/or it may be a mutation wherein the mutant HvHRT gene of said barley plant encodes a mutant HvHRT protein comprising a W431stop mutation of SEQ ID NO: 2 of WO 2019/129739. Said mutation of the HvHRT gene may for example also be a G→A mutation of the nucleotide 510 of the HvHRT coding sequence of SEQ ID NO:1 of WO 2019/129739, and/or a mutation, wherein the mutant HvHRT gene encodes a mutant HvHRT protein having a W170stop mutation of SEQ ID NO: 2 of WO 2019/129739. Said mutation of the HvHRT gene may for example also be a G→A mutation of the nucleotide 1113 of the HvHRT coding sequence of SEQ ID NO:1 of WO 2019/129739 and/or it may be a mutation wherein the mutant HvHRT gene of said barley plant encodes a mutant HvHRT protein comprising a W371stop mutation of SEQ ID NO: 2 of WO 2019/129739. Mutation of HvHRT may increase alpha-amylase in the barley kernel. Increased alpha amylase activity in malting increases the starch degradation and the availability of fermentable sugars in the kernel.

In addition to the mutation in the HvLDI gene described herein, the barley plants of the invention may also comprise any of the mutations leading to increased alpha-amylase activity described in international patent application WO 2019/129739. In particular, the barley plants of the invention may comprise a mutation in the HvHBL12 gene (SEQ ID NO: 5 in WO 2019/129739 and NCBI accession numbers AK376953.1 and AK361212.1) leading to a loss of HBL12 function. Said mutation in the HvHBL12 gene may for example be any of the mutations in the HvHBL12 gene described in international patent application WO 2019/129739. For example the barley plant may comprise a gene encoding a mutant HvHBL 12 gene encoding a mutant HvHBL 12 protein lacking at least i) amino acids 26 to 79 of SEQ ID NO:6 in WO 2019/129739; or ii) amino acids 81 to 122 of SEQ ID NO:6 or iii) amino acids 228 to 250 of SEQ ID NO:6 in WO 2019/129739. The same mutations may occur in one of the polymorphisms of SEQ ID NO:6 in WO 2019/129739 namely polymorphisms N141 D, M142V or E184D. Alternatively, the barley plant may comprises a premature stop codon in the HvHDL12 gene. In particular said barley plant may comprise a G→A mutation of the nucleotide 684 of the HvHBL12 coding sequence of SEQ ID NO:5 in WO 2019/129739 or any of the above mentioned polymorphs thereof, this encodes a mutant HvHBL 12 protein comprising a W228stop mutation of SEQ ID NO: 6 in WO 2019/129739. Barley kernels with lack of HvHBL function has been shown to have a higher alpha-amylase activity.

In addition to the mutation in the HvLDI gene described herein, the barley plants of the invention may also comprise any of the mutations leading to increased alpha-amylase activity described in international patent application WO 2019/129739. In particular, the barley plants of the invention may comprise a mutation in the WKRY38 gene (SEQ ID NO: 10 in WO 2019/129739 or NCBI accession number AJ536667.1 or AK360269.1 or AY541586.1) leading to a loss of WKRY38 function. Said mutation in the WKRY38 gene may for example be any of the mutations in the WKRY38 gene described in international patent application WO 2019/129739. In particular said barley plant may comprise a G→A mutation of the nucleotide 600 of the HvWRKY38 coding sequence of SEQ ID NO:10 in WO 2019/129739. Barley kernels with lack of WKRY38 function has been shown to have a higher alpha-amylase activity.

In addition to the mutation in the HvLDI gene described herein, the barley plants of the invention may also comprise anthocyanin- and proanthocyanidin-free mutant (an ant mutation), such as any of the ant mutations described by Himi et al., 2012 or Jende-Strid, 1993. In particular, the ant mutation may be a mutation of the Hvmyb10 gene (Gen Bank accession nr. AB645844), for example a non-synonymous mutation of the Hvmyb10, preferably any of the mutations in the Hvmyb10 gene found in an ant28 mutants. Thus, the ant mutation may be a G→A mutation of nucleotide 51 of the coding region of wild type Hvmyb10 or a G→A mutation of nucleotide 558 of the coding region of wild type Hvmyb10 as described in Himi et al., 2012. In particular ant28 mutants have a reduced level of grain dormancy.

The barley plants of the invention may also comprise a combination of the additional mutations mentioned above. Potential combinations of gene mutations are illustrated in the table below showing 8 examples of different barley plants, wherein “Mut” indicates that the plant comprises any of the mutations described herein in the indicated gene.

| | LDI | LOX-1 | MMT | CsIF6 | HRT | HBL12 | WRKY38 | Myb10 |
|---|---|---|---|---|---|---|---|---|
| Ex. 1 | Mut | Mut | Mut | | | | | Mut |
| Ex. 2 | Mut | Mut | Mut | Mut | | | | Mut |
| Ex. 3 | Mut | Mut | Mut | Mut | | Mut | | Mut |
| Ex. 4 | Mut | Mut | Mut | Mut | | | Mut | Mut |
| Ex. 5 | Mut | Mut | Mut | Mut | Mut | | | Mut |
| Ex. 6 | Mut | Mut | Mut | | | Mut | | Mut |
| Ex. 7 | Mut | Mut | Mut | | | | Mut | Mut |
| Ex. 8 | Mut | Mut | Mut | | Mut | | | Mut |

Particularly described is a barley plant with an LDI mutation of the present invention and a loss of function mutation in LOX-1 and MMT and Myb10 (illustrated as Ex. 1 in the first line in the above table), as well as a barley plant with an LDI mutation of the present invention and loss of function mutations in LOX-1 and MMT and CsIF6 and Myb10 (illustrated as Ex. 2 in the second line in the table above).

Barley plants comprising more than one mutation may be produced by any useful methods. For example, said one or more additional mutation may be introduced into a barley plant carrying a mutation in the HvLDI gene or alternatively, a mutation in the HvLDI gene as described herein may be introduced into a barley plant already carrying the additional mutation. Barley plants carrying a specific desired mutation may be prepared and identified essentially as described in international patent application WO 2018/001884 using primers and probes designed to identify the particular mutation.

Alternatively, said barley plants may be prepared by crossing barley plants carrying a mutation in the HvLDI gene with a barley plant carrying one or more of the additional mutations, e.g. any of the barley plants described in or deposited in relation to international patent applications WO 2005/087934, WO 2010/075860, WO 2010/063288, WO 2019/129736 or WO 2019/129739 or described in Himi et al., 2012.

### Plant Products

The invention also provides plant products prepared from a barley plant carrying a mutation in the HvLDI gene of the invention, e.g. any of the barley plants, or parts thereof, described herein.

The plant product may be any product prepared from a barley plant, for example a food, a feed or a beverage. Thus the plant product may be any of the beverages described herein below in the section “Beverage and method of production thereof”. The plant product may also be an aqueous extract of the barley plant and/or of malt of said barley plant, for example the plant product may be wort. Said aqueous extract may for example be prepared as described herein below in the section “Aqueous extract and methods of production thereof”.

In one embodiment the plant product may be malt, e.g. any of the malts described herein below in the section “Malt and methods of production thereof” or a malt based product, such as malt based beverages. Although the primary use of malt is for beverage production, it can also be utilized in other industrial processes, for example as an enzyme source in the baking industry, or in the food industry as a flavouring and colouring agent, e.g. in the form of malt or malt flour or indirectly as a malt syrup, etc. Thus, the plant product according to the invention may be any of the aforementioned products.

In another aspect, the plant products according to the invention comprise, or even consist of syrup, such as a barley syrup, or a barley malt syrup. The plant product may also be an extract of barley or malt. Thus, the plant product may be wort.

### Malt and Methods of Production Thereof

The invention also provides malt prepared from a barley plant carrying a mutation in the HvLDI gene of the invention, for example any of the barley plants described herein.

Malt may be prepared by malting, i.e. by germination of steeped barley grains in a process taking place under controlled environmental conditions. The germination may optionally be followed by a drying step. Said drying step may preferably be kiln drying of the germinated grains at elevated temperatures.

Thus, a method of malting according to the present invention preferably comprises the steps of:
a) providing grains of a barley plant, or part thereof, of the invention;
b) steeping and germinating said barley grains under predetermined conditions;
c) optionally, drying said germinated barley grains.

The barley of the present invention is particularly suited for green malt processes, i.e. a malting process where the malt is not kiln dried prior to mashing. The barley of the present invention is also particularly suited for short malting processes, e.g. the process described below, due to the high limit dextrinase levels.
a) In one embodiment the malt is prepared according to the following process: Barley grains, which optionally have been cleaned are steeped (incubated) in an aqueous solution, preferably in water for a time period of in the range of 4 to 24 h, preferably for in the range of 5 to 15 h, more preferably for in the range of 5-10 h. During this period the aqueous solution comprising the grains is aerated. Aeration may increase xygen levels in the aqueous solution and/or loosen the barley grains and/or to aid in avoiding dry pockets amongst the barley grain. The steeping may be performed at any useful temperature, preferably at temperature in the range of 20to 28° C., more preferably at approximately 25° C.;
b) Aqueous solution is drained off and the grain is subjected to an air rest for in the range of 5 to 30 h, preferably for in the range of 8 to 24 h, more preferably for in the range of 8 to 16 h. Preferably, the grain is aerated during the air rest. Preferably, a constant temperature in the range of of 20-28° C. is maintained in the germinating grain, e.g. by controlling the temperature of the gas used for aeration;
c) The barley grain is incubated in aqueous solution for in the range of 2 to 24 h, preferably for in the range of 2 to 15 h, more preferably for in the range of 2-10 h with aeration, e.g. to mix the grain. Preferably the water is kept at a temperature in the range of 20 to 28° C. , such as around 25° C.; and
d) Aqueous solution is drained off and the grain is subjected to an air rest phase for in the range of 5 to 30 h, preferably for in the range of 8 to 20 h. Preferably, the temperature is kept in a range of 20 to 28° C., e.g. by controlling the temperature of the gas used for aeration.
e) The germination process is preferably completed within 48 to 72 h, preferably within 48 to 60 h, even more preferably within 48-56 h. Preferably, the germinated grains have a water content of at least 20% throughout the whole process subsequent to step a. The germinated grans may be directly employed in further processes as green malt.

In one embodiment, said malt may be prepared by a method involving incubation of grains in water under aeration and without kiln drying. Such malt may also be referred to as “flex-malt” herein. In particular, flex-malt may be prepared as described in WO 2018/001882 and WO 2019/129724. In particular as described in the section “Germination” page 14 to page 22, “Heat treatment” page 22 to page 24 and “Example 1” page 56 to page 57 in WO 2018/001882, as well as “Germination” page 14 to 22, “Heat treatment” page 22 to page 24 and “Example 1” page 56 to page 57 in WO 2019/129724, hereby incorporated by reference.

A method of preparing “flex-malt” is described herein below in Example 5. Germination is normally initiated at the time point wherein the grains are in contact with water, such as at a time point at which barley grains with a water content of less than 15% is contacted with sufficient water to initiate germination.

In one embodiment, germination is initiated when the grains are aerated from beneath with varying levels of atmospheric air for different time period, during which the grain moisture content is raised.

The grains of barley plant of the invention have shown to be particular useful when the germination process is shortened. Thus, grains of barley plants of the invention are indeed useful in malting methods wherein the germination time is shortened. In one embodiment, the step of steeping and germination of grains of the invention is performed for at the most 4 days, such as for the most 3 days.

In another embodiment, malt may be prepared by conventional malting, wherein the steeping process and germination process are performed in two separate steps. Thus, steeping may be performed by any conventional method known to the skilled person. One non-limiting example involves steeping at a temperature in the range of 10 to 25° C. with alternating dry and wet conditions. During steeping, for example, the cereal kernels may be incubated wet for in the range of 30 min to 3 h followed by incubation dry for in the range of 30 min to 3 h and optionally repeating said incubation scheme in the range of 2 to 5 times. The final water content after steeping may, for example, be in the range of 40 to 50%. Germination of grains may be performed by any conventional method known to the skilled person. One non-limiting example involves germination at a temperature in the range of 10 to 25° C., optionally with changing temperature in the range of 1 to 6 days.

Optionally, the kiln drying may be performed at conventional temperatures, such as at least 75° C., for example in the range of 80 to 90° C., such as in the range of 80 to 85° C.

Thus, the malt may, for example be produced by any of the methods described by Hough et al. (1982). However, any other suitable method for producing malt may also be used with the present invention, such as methods for production of specialty malts, including, but not limited to, methods of roasting the malt.

Malt may be further processed, for example by milling. Thus, the plant product according to the invention may be any kind of malt, such as unprocessed malt or milled malt, such as flour. Milled malt and flour thereof comprise chemical components of the malt and dead cells that lack the capacity to re-germinate.

The milling may be performed in a dry state, i.e. the malt is milled while dry, or the milling may be performed in a wet state, i.e. the malt is milled while wet.

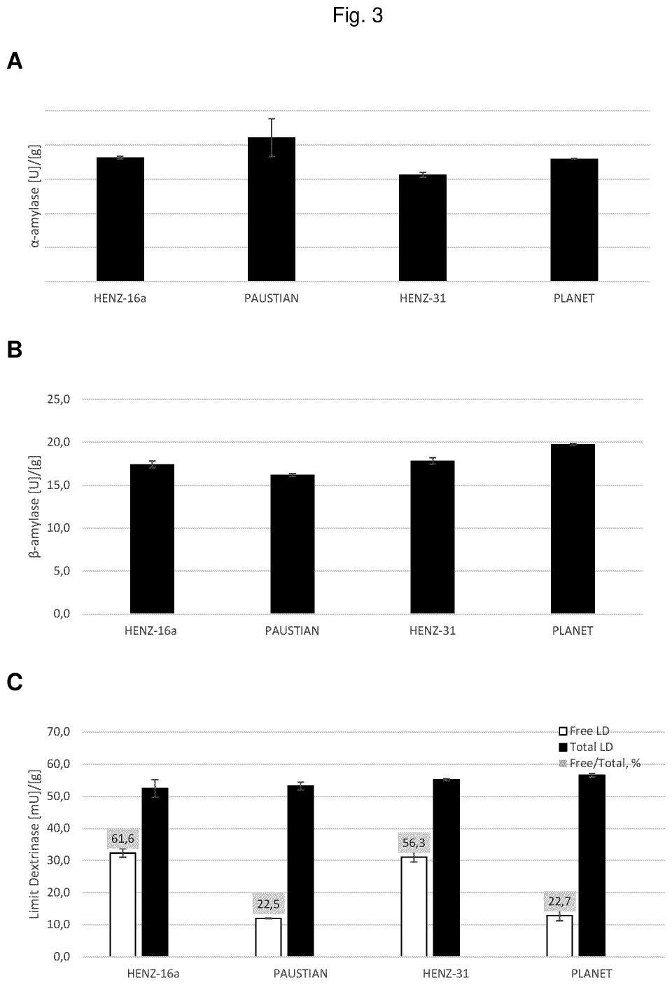

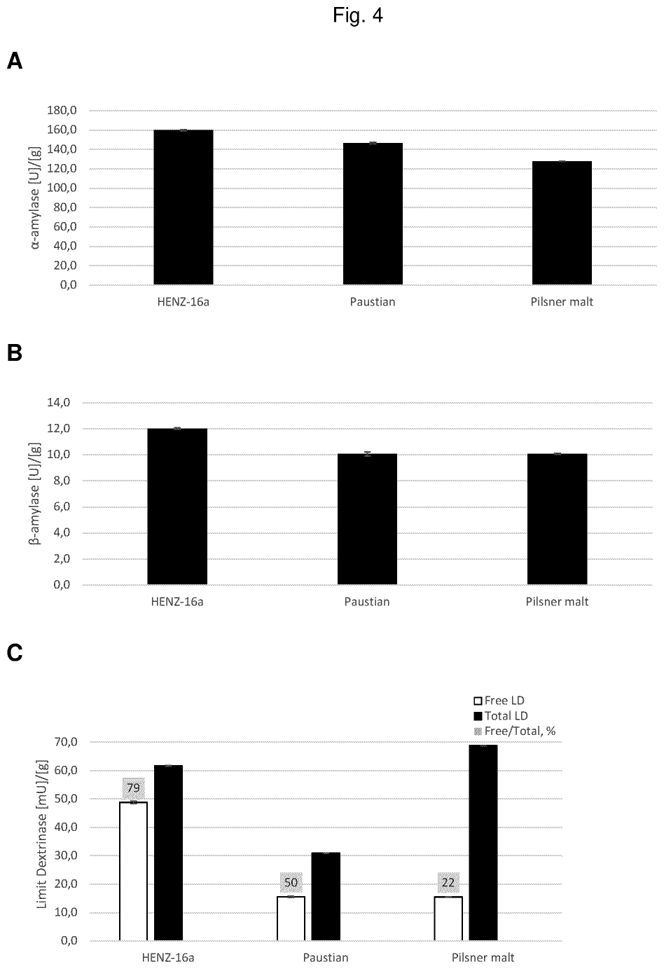

An advantage of malt prepared from barley plants, or parts thereof, carrying a mutation in the HvLDI gene of the invention is that said malt have a high level of free HvLD activity when compared to malt from wt barely plants without the HvLDI mutation but otherwise of the same genotype. The high level of free HvLD activity renders the malt advantageous, for several reasons. Increased free HvLD activity is useful in malting processes, wherein the germination time is shortened. In particular FIG. 3 shows that malt from barley of the invention (HENZ-16a and 31) when used in the flex-malt process outperforms wt barley such as Paustian in the conventional malting process (FIG. 4) in that the free/total ratio of limit dextrinase is still improved with 61.6 and 56.3%, for the HENZ variant in the flex malt process over 50% for Paustian in the conventional process. Moreover, increased free HvLD activity leads to increased degradation of starch into fermentable sugars and thus wort prepared from grains and/or malt from said barley plants may be characterized by a higher content of fermentable sugars. Thus, by using malt of the invention, the need for addition of exogenous limit dextrinase or pullulanase during mashing may be reduced or even completely abolished. Furthermore, fermenting an aqueous extract containing a high content of fermentable sugars is a benefit during brewing, since it increases the amount of beer produced per amount of grains used and increase the ABV % (alcohol by volume) pr. hectolitre per grain weight.

### Aqueous Extract and Method of Production Thereof

The invention provides barley based beverages as well as methods of preparing the same, wherein the barley plant carrying a mutation in the HvLDI gene of the invention.

Frequently, methods for preparing a barley based beverage comprise a step of preparing an aqueous extract of grains of the barley plants of the invention and/or of malts prepared from barley plants of the invention and optionally one or more adjuncts.

In one embodiment, the aqueous extract is prepared from grains of the barley plant of the invention and/or malt prepared from barley plants of the invention. In another embodiment, the aqueous extract is prepared from a mixture of grains of the barley plant of the invention and/or malt prepared from barley plants of the invention and grains of a wt barley plant and/or malt prepared form grains of a wt barley plant and optionally one or more adjuncts. In some embodiments, at least 10%, such as at least 20%, such as at least 30%, such as at least 40%, such as at least 50%, such as at least 60%, such as at least 70%, such as at least 80%, such as at least 90%, such as 100% of the barley grains and/or malt used to prepare the aqueous extract may be barley grains of a barley plant according to the invention or malt prepared from a barley plant of the invention and optionally one or more adjuncts.

In one embodiment the malt used in the aqueous extract is a green malt or flex malt as described in the section “Malt and methods of production thereof”, specifically green malt may bemilled in the wet state. Specifically, the green malt/flex malt never had a water content below 20% prior to milling and mashing, and has not been subjected to kilning.

The aqueous extract may, in general, be prepared by incubating barley flour and/or malt flour in water or in an aqueous solution. In particular, the aqueous extract may be prepared by mashing.

In general said aqueous solution may be water, such as tap water to which one or more additional agents may be added. The additional agents may be present in the aqueous solution from the onset or they may be added during the process of preparing an aqueous extract. Said additional agents may be enzymes. Thus, the aqueous solution may comprise one or more enzymes. Said enzymes may be added to the aqueous solution from the onset, or subsequently, during the process.

Said enzymes may, for example, be one or more hydrolytic enzymes. Suitable enzymes include lipases, starch degrading enzymes (e.g. amylases), glucanases [preferably (1-4)- and/or (1,3;1,4)-beta-glucanases], and/or xylanases (such as arabinoxylanases), and/or proteases, or enzyme mixtures comprising one or more of the aforementioned enzymes, e.g. Cereflo, Ultraflo, or Ondea Pro (Novozymes). For example, the aqueous solution may comprise one or more hydrolytic enzymes selected from the group consisting of alpha-amylase, beta-amylase, limit dextrinase, pullulanase, β-glucanase (e.g. endo-(1,3;1,4)-beta-glucanase or endo-1,4-beta-glucanase), xylanase (e.g. endo- or exo-1,4-xylanase, an arabinofuranosidase or a ferulic acid esterase), glucoamylase- and protease.

One advantage of the barley plants of the invention is the amount of high free HvLD activity in the grains of said barley plants or in malt prepared from said barley plants. Sometimes, when mashing barley grains and/or malt, limit dextrinase or other enzymes capable of catalysing hydrolysis of alpha-1,6 linkages, such as pullulanase may be added in order to advance starch hydrolysis by releasing straight chain dextrins from amylopectin-derived branched dextrins. It is thus one advantage of the present invention that the need for addition of exogenous limit dextrinase or pullulanase is reduced or even abolished, and an adequate level of fermentable sugars in the extract can still be obtained. In some embodiments of the invention, it may even be possible to prepare the aqueous extract without addition of exogenous limit dextrinase or pullulanase.

Said additional agents, preferably of food grade quality, may also be a salt, for example CaCl₂, or an acid, for example H₃PO₄.

The aqueous extract is generally prepared by incubation of the barley flour and/or malt flour in the aqueous solution at one or more predetermined temperature(s). Said predetermined temperature may also be referred to as “mashing temperature” herein. Said mashing temperatures may for example be conventional temperatures used for mashing. The mashing temperature is in general either kept constant (isothermal mashing), or gradually increased, for example increased in a sequential, step-wise manner. In either case, soluble substances in the barley grains and/or malt are liberated into said aqueous solution thereby forming an aqueous extract.

The mashing temperature(s) are typically temperature(s) in the range of 30 to 90° C., such as in the range of 40 to 85° C., for example in the range of 50 to 85° C. In particular, a relatively low mashing-in temperature may be used, e.g. a temperature in the range of 50-60° C.

Subsequent to incubation in the aqueous solution in e.g. a mashing vessel, the aqueous solution may be transferred to another container, e.g. a lauter tun and incubated for additional time at elevated temperature.

Non-limiting examples of useful mashing protocols can be found in the literature of brewing, e.g. in Hough et al. (supra).

Mashing (i.e. incubation of the barley flour and/or malt flour in aqueous solution) can occur in the presence of adjuncts, which is understood to comprise any carbohydrate source other than malt, such as, but not limited to, barley, barley syrups, or maize, or rice—either as whole kernels or processed products like grits, syrups or starch. All of the aforementioned adjuncts may be used principally as an additional source of extract (syrups are typically dosed during wort heating). The requirements for processing of the adjunct in the brewery depend on the state and type of adjunct used, and in particular on the starch gelatinisation or liquefaction temperatures.

After incubation in the aqueous solution, the aqueous extract may typically be separated, e.g. through filtration into the aqueous extract and residual non-dissolved solid particles, the latter also denoted “spent grain”. Filtering may for example be performed in a lauter tun. Alternatively, the filtering may be filtering through a mash filter. The aqueous extract thus obtained may also be denoted “first wort”. Additional liquid, such as water may be added to the spent grains during a process also denoted sparging. After sparging and filtration, a “second wort” may be obtained. Further worts may be prepared by repeating the procedure. Thus, the aqueous extract may be wort, e.g. a first wort, a second wort, a further wort or a combination thereof.

One advantage of aqueous extracts prepared from barley plants carrying a mutation in the HvLDI gene of the invention may be that they contain a high level of fermentable sugars.

In one embodiment, said aqueous extract prepared from grains and/or malt of barley plants according to the present invention have an increased concentration of total fermentable sugars compared to an aqueous extract of barley plants carrying a HvLDI gene encoding a wt HvLDI, but otherwise of the same genotype, when prepared by under the same conditions.

In another embodiment, said aqueous extract prepared from malt prepared form barley plants according to the present invention have at least 5% more total fermentable sugars, such as at least 6% more total fermentable sugars, such as at least 7% more total fermentable sugars compared to an aqueous extract of barley plants carrying a HvLDI gene encoding a wt HvLDI, but otherwise of the same genotype, when prepared under the same conditions.

In another embodiment, said aqueous extract prepared from malt prepared form barley plants according to the present invention have at least 10% more glucose, fructose and/or maltotriose compared to an aqueous extract of barley plants carrying a HvLDI gene encoding a wt HvLDI, but otherwise of the same genotype, when prepared under the same conditions.

### Beverages and Methods of Production Thereof

The present invention also provides barley based beverages and methods of producing such beverages, wherein the barley plant carries a mutation in the HvLDI gene of the invention.

Said beverage may be an alcoholic barley based beverages or non-alcoholic barley based beverages. Alcoholic barley based beverages may for example be beer or a distilled alcohol.

Said beer may be any kind of beer, for example lager or ale. Thus, the beer may for example be selected from the group consisting of Altbier, Amber ale, Barley wine, Berliner Weisse, Bière de Garde, Bitter, Blonde Ale, Bock, Brown ale, California Common, Cream Ale, Dortmunder Export, Doppelbock, Dunkel, Dunkelweizen, Eisbock, Fruit Iambic, Golden Ale, Gose, Gueuze, Hefeweizen, Helles, India pale ale, Kolsch, Lambic, Light ale, Maibock, Malt liquor, Mild, Märzenbier, Old ale, Oud bruin, Pale ale, Pilsener, Porter, Red ale, Roggenbier, Saison, Scotch ale, Steam beer, Stout, Schwarzbier, lager, Witbier, Weissbier and Weizenbock. The beer may also be a low-alcohol or non-alcoholic beer (also known as “alcohol free beer” or afb)

Said distilled alcohol may be any kind of distilled alcohol. In particular the distilled alcohol may be based on a cereal, e.g. a malted cereal, e.g. a barley malt. Non-limiting examples of such distilled alcohol include whiskey and vodka.

The beverage may be a non-alcoholic beverage, such as a non-alcoholic barley based beverage, e.g. non-alcoholic beer or non-alcoholic malt beverages, such as maltina or noussy.

The beverage may for example be prepared by a method comprising the steps of:
a. providing grains of a barley plant according to the invention and/or malt prepared from grains of a barley plant according to the invention and/or an aqueous extract prepared from grains and/or malt of a barley plant according to the invention;
b. processing said aqueous extract into a beverage.

The aqueous extract may be boiled with or without hops where after it may be referred to as boiled wort. First, second and further worts may be combined, and thereafter subjected to boiling. The aqueous extract may be boiled for any suitable amount of time, e.g. in the range of 60 min to 120 min.

Step (a) may in particular comprises fermentation of said aqueous extract, e.g. by fermentation of wort. Thus, the beverage may be prepared by fermentation of the aqueous extract with yeast.

Once the aqueous extract has been prepared it may be processed into beer by any method including conventional brewing methods. Non-limited descriptions of examples of suitable methods for brewing can be found, for example, in publications by Hough et al. (1982). Numerous, regularly updated methods for analyses of barley and beer products are available, for example, but not limited to, American Association of Cereal Chemists (1995), American Society of Brewing Chemists (1992), European Brewery Convention (1998), and Institute of Brewing (1997). It is recognized that many specific procedures are employed for a given brewery, with the most significant variations relating to local consumer preferences. Any such method of producing beer may be used with the present invention.

The first step of producing beer from the aqueous extract preferably involves boiling said aqueous extract as described herein above, followed by a subsequent phase of cooling and optionally whirlpool rest. One or more additional compounds may be added to the aqueous extract, e.g. one or more of the additional compounds described below in the section “Additional compounds”. After being cooled, the aqueous extract may be transferred to fermentation tanks containing yeast, e.g. brewing yeast, such as S. pastorianus or S. cerevisiae. The aqueous extract may be fermented for any suitable time period, in general in the range of 1 to 20 days, such as 1 to 10 days. The fermentation is performed at any useful temperature e.g. at a temperature in the range of 10 to 20° C. The methods may also comprise addition of one or more enzymes, e.g. one or more enzymes may be added to the wort prior to or during fermentation. In particular, said enzyme may be a proline-specific endoprotease. A non-limiting example of a proline-specific endoprotease is “Brewer's Clarex” available from DSM. In other embodiments, no exogenous enzymes are added during the methods.

During the several-day-long fermentation process, sugar is converted to alcohol and CO₂ concomitantly with the development of some flavour substances. The fermentation may be terminated at any desirable time, e.g. once no further drop in % P is observed.

Subsequently, the beer may be further processed, for example chilled. It may also be filtered and/or largered—a process that develops a pleasant aroma and a less yeast-like flavour. Additives may also be added. Furthermore, CO₂ may be added. Finally, the beer may be pasteurized and/or filtered, before it is packaged (e.g. transferred to containers or kegs, bottled or canned). The beer may also be pasteurized by standard methods.

### Additional Compounds

The methods of the invention may comprise the step of adding one or more additional compounds. Said additional compounds may for example be a flavor compound, a preservative, a functional ingredient, a color, a sweetener, a pH regulating agent or a salt. The pH regulating agent may for example be a buffer or an acid, such as phosphoric acid.

Functional ingredients may be any ingredient added to obtain a given function. Preferably, a functional ingredient renders the beverage healthier. Non-limiting examples of functional ingredients includes vitamins or minerals.

The preservative may be any food grade preservative, for example it may be benzoic acid, sorbic acid, sorbates (e.g. potassium sorbate), sulphites and/or salts thereof.

The additional compound may also be CO₂. In particular, CO₂ may be added to obtain a carbonated beverage.

The flavour compound to be used with the present invention may be any useful flavour compound. The flavour compound may for example be selected from the group consisting of aromas, plant extracts, plant concentrates, plant parts and herbal infusions. In particular, the flavour compounds may be hops.

### Methods of Preparing a Barley Plant Carrying a Mutation in the HvLDI Gene of the Invention

Barley plants carrying a mutation in the HvLDI gene of the invention may be prepared in any useful manner.

For example, such barley plants can be prepared by a method comprising the steps of:
a. providing barley grains; and
b. randomly mutagenizing said barley grains,
c. selecting barley grains or parts thereof carrying a mutated HvLDI gene encoding a mutant HvLDI polypeptide carrying one of the following mutations
   i. a missense mutation resulting in a change from Pro to a different amino acid in one or more loop regions of HvLDI, wherein the loop regions correspond to amino acids corresponding to position 25 to 44 and amino acids corresponding to position 56 to 62 and amino acids corresponding to position 77 to 78 and amino acids corresponding to position 91 to 111 and amino acids corresponding to position 124 to 147 of SEQ ID NO:1; or
   ii. a missense mutation resulting in a change from an acidic amino acid to a non-acidic amino acid in one or more alpha helix regions of wt HvLDI, wherein the alpha helix regions correspond to amino acids 45 to 55 and amino acids 63 to 76 and amino acids 79 to 90 and/or amino acids 112 to 123 of SEQ ID NO:1.

Such methods may also include one or more steps of reproducing said barley plants/barley grains in order to obtain multiple barley plants/grains each carrying said mutation.

In particular, barley plants carrying a particular mutation in HvLDI gene may be prepared and identified essentially as described in international patent application WO 2018/001884 using primers and probes designed to identify a mutation in the HvLDI gene. The genomic sequence of HvLDI can be retrieved from public databases by blast searches using the coding sequences of SEQ ID NO:2, or it may be found under the Gen Bank accession number DQ285564.1.

Barley plants carrying a mutation in the HvLDI gene may also be prepared using various site directed mutatgenesis methods, which for example can be designed based on the sequence of the coding sequence of SEQ ID NO:2. In one embodiment, the barley plant is prepared using any one of CRISPR, a TALEN, a zinc finger, meganuclease, and a DNA-cutting antibiotic as described in WO 2017/138986. In one embodiment, the barley plant is prepared using CRISPR/cas9 technique, e.g. using RNA-guided Cas9 nuclease. This may be done as described in Lawrenson et al., Genome Biology (2015) 16:258; DOI 10.1186/s13059-015-0826-7 except that the single guide RNA sequence is designed based on the gene sequence of HvLDI. In one embodiment, the barley plant is prepared using a combination of both TALEN and CRISPR/cas9 techniques, e.g. using RNA-guided Cas9 nuclease. This may be done as described in Holme et al., 2017) except that the TALEN and single guide RNA sequence are designed based on the genes sequences provided herein.

In one embodiment, the barley plant is prepared using homology directed repair, a combination of a DNA cutting nuclease and a donor DNA fragment. This may be done as described in Sun et al., 2016 except that the DNA cutting nuclease is designed based on the genes sequences provided herein and the donor DNA fragment is designed based on the coding sequence of the mutated barley variant provided herein.

In one embodiment of the invention, the objective is to provide agronomical useful barley plants carrying a mutation in the HvLDI gene. In addition to the mutation in the HvLDI gene, there are additional factors which also may be considered in the art of generating a commercial barley variety useful for malting and/or brewing and/or as base for beverages, for example kernel yield and size, and other parameters that relate to malting performance or brewing performance. Since many—if not all—relevant traits have been shown to be under genetic control, the present invention also provides modern, homozygous, high-yielding malting cultivars, which may be prepared from crosses with the barley plants that are disclosed in the present publication. The skilled barley breeder will be able to select and develop barley plants, which—following crossings with other barley plants—will result in superior cultivars. Alternatively, the breeder may utilize plants of the present invention for further mutagenesis to generate new cultivars carrying additional mutations in addition to the mutation of the HvLDI gene.

The invention also comprise barley plants carrying a mutation in the HvLDI gene prepared from plant breeding method, including methods of selfing, backcrossing, crossing to populations, and the like. Backcrossing methods can be used with the present invention to introduce into another cultivar the mutation of the HvLDI gene.

A way to accelerate the process of plant breeding comprises the initial multiplication of generated mutants by application of tissue culture and regeneration techniques. Thus, another aspect of the present invention is to provide cells, which upon growth and differentiation produce barley plants carrying the mutation of the HvLDI gene. For example, breeding may involve traditional crossings, preparing fertile anther-derived plants or using microspore culture.

### Items

The invention may further be defined by the following items.
1. A barley plant, or a part thereof, wherein said barley plant carries a mutation in the HvLDI gene, wherein said mutated HvLDI gene encodes a mutant HvLDI polypeptide, wherein the mutation is one of the following mutations
   a. a missense mutation resulting in a change from a proline to a different amino acid in one or more loop regions of the mutant HvLDI polypeptide, wherein the loop regions are selected from the group consisting of amino acids corresponding to position 25 to 44 and amino acids corresponding to position 56 to 62 and amino acids corresponding to position 77 to 78 and amino acids corresponding to position 91 to 111 and amino acids corresponding to position 124 to 147 of SEQ ID NO:1; or
   b. a missense mutation resulting in a change from a negatively charged amino acid to a non-negatively charged amino acid in one or more alpha helix regions of the mutant HvLDI polypeptide, wherein the alpha helix regions are selected from the group consisting of amino acids corresponding to position 45 to 55 and amino acids corresponding to position 63 to 76 and amino acids corresponding to position 79 to 90 and amino acids corresponding to position 112 to 123 of SEQ ID NO:1.
2. The barley plant or part thereof according item 1, wherein said mutant HvLDI polypeptide is at least 90% identical to the mature wt HvLDI polypeptide of SEQ ID NO: 1.
3. The barley plant or part thereof according to any one of item 1 or 2, wherein said mutant HvLDI polypeptide is 98% identical to a mature wt HvLDI polypeptide listed in table 1A and 1B or natural variants thereof.
4. The barley plant or part thereof according to any one of items 1 to 3, wherein said mutated HvLDI gene encodes a mutant HvLDI polypeptide, wherein the loop regions are selected from the group consisting of amino acids corresponding to position 56 to 62 and amino acids corresponding to position 77 to 78 and amino acids corresponding to position 91 to 111 of SEQ ID NO:1.
5. The barley plant or part thereof according to any one of the preceding items, wherein said mutated HvLDI gene encodes a mutant HvLDI polypeptide, wherein said mutant HvLDI polypeptide comprises a substitution of a proline in one or more of the loop regions of wt HvLDI to a polar amino acid.
6. The barley plant or part thereof according to any one of the preceding items, wherein said mutated HvLDI gene encodes a mutant HvLDI polypeptide, wherein said mutant HvLDI polypeptide comprises a comprises a substitution of a proline in one or more of the loop regions of HvLDI to serine.
7. The barley plant or part thereof according any one of the preceding items, wherein said mutated HvLDI gene encodes a mutant HvLDI polypeptide, wherein said mutant HvLDI polypeptide comprises a substitution of a proline at a position corresponding to position 60 of SEQ ID NO:1 to a different amino acid.
8. The barley plant or part thereof according any one of the preceding items, wherein said mutated HvLDI gene encodes a mutant HvLDI polypeptide, wherein said mutant HvLDI polypeptide consists of LDI of SEQ ID NO:1 having a substitution of the proline at position 60 of SEQ ID NO:1 to a different amino acid.
9. The barley plant or part thereof according to item 7 or 8, wherein the proline is substituted with a serine.
10. The barley plant or part thereof according to any one of the preceding items, wherein said mutated HvLDI gene encodes a mutant HvLDI polypeptide, wherein said mutant HvLDI polypeptide comprises a substitution of a proline at amino acid position 60 of SEQ ID NO:1 to a serine.
11. The barley plant or part thereof according to any one of the preceding items, wherein the mutant HvLDI polypeptide comprises or consists of the amino acid sequence from position 25 to 142 of SEQ ID NO: 3 or from position 25 to 147 of SEQ ID NO: 3.
12. The barley plant or part thereof according to any one of the preceding items, wherein the mutant HvLDI polypeptide comprises or consists of the amino acid sequence from position 25 to 142 of SEQ ID NO: 4 or from position 25 to 147 of SEQ ID NO: 4.
13. The barley plant or part thereof according to any one of the preceding items, wherein said mutated HvLDI gene encodes a mutant HvLDI polypeptide comprising a substitution of a negatively charged amino acid in one or more of the alpha helix regions of HvLDI to a positively charged amino acid.
14. The barley plant or part thereof according to any one of the preceding items, wherein said mutated HvLDI gene encodes a mutant HvLDI polypeptide, wherein said mutant HvLDI polypeptide comprises a substitution of a negatively charged amino acid in one or more of the alpha helix regions of HvLDI to a lysine.
15. The barley plant or part thereof according to any one of items 13 or 14, wherein said negatively charged amino acid is glutamic acid (Glu).
16. The barley plant or part thereof according any one of the preceding items, wherein said mutated HvLDI gene encodes a mutant HvLDI polypeptide, wherein said mutant HvLDI polypeptide comprises a substitution of a glutamic acid at a position corresponding to position 68 of SEQ ID NO:1 to a non-negatively charged amino acid.
17. The barley plant or part thereof according any one of the preceding items, wherein said mutated HvLDI gene encodes a mutant HvLDI polypeptide, wherein said mutant HvLDI polypeptide consists of LDI of SEQ ID NO:1 having a substitution of the glutamic acid at position 68 of SEQ ID NO:1 to a non-negatively charged amino acid.
18. The barley plant or part thereof according to any one of the preceding items, wherein said mutated HvLDI gene encodes a mutant HvLDI polypeptide, wherein said mutant HvLDI polypeptide comprises a substitution of a glutamic acid at amino acid position 68 of SEQ ID NO:1 to lysine.
19. The barley plant or part thereof according to any one of the preceding items, wherein the mutant HvLDI polypeptide comprises or consists of the amino acid sequence from position 25 to 142 of SEQ ID NO: 6 or from position 25 to 147 of SEQ ID NO: 6.
20. The barley plant or part thereof according to any one of the preceding items, wherein grains of said barley plant have a free HvLD activity at least 20% higher compared to the free HvLD activity measured in grains of a barley plant carrying a HvLDI gene encoding a wt HvLDI polypeptide, but otherwise of the same genotype, when cultivated under the same conditions.
21. The barley plant or part thereof according to any one of the preceding items, wherein said germinated grains have a free HvLD activity at least 20 % higher compared to the free HvLD activity measured in germinated grains of barley plants carrying a HvLDI gene encoding a wt HvLDI polypeptide, but otherwise of the same genotype, when prepared under the same conditions.
22. The barley plant or part thereof according to any one of the preceding items, wherein malt prepared from said barley plant have a free HvLD activity at least 20% higher compared to the free HvLD activity measured in malt of barley plants carrying a HvLDI gene encoding a wt HvLDI polypeptide but otherwise of the same genotype, when prepared under the same conditions.
23. The barley plant or part thereof according to any one of the preceding items, wherein said free HvLD activity is at least 50% higher, such as at least 100% higher, preferably at least 140% higher compared to the free HvLD activity measured in malt of barley plants carrying a HvLDI gene encoding a wt HvLDI polypeptide but otherwise of the same genotype, when prepared under the same conditions.
24. The barley plant or part thereof according to any one of the preceding items, wherein grains or germinated grains or malt from said barley plant have a free/total % HvLD activity at least 20% higher compared to the free/total % HvLD activity measured in grains or germinated grains or malt, respectively, of barley plants carrying a HvLDI gene encoding a wt HvLDI polypeptide, but otherwise of the same genotype, when cultivated and prepared under the same conditions.
25. The barley plant or part thereof according to any one of the preceding items, wherein said barley plant carries one or more mutations in the HvLDI gene selected from the group consisting of:
   i. a mutation of nucleotide C to T at the position corresponding to nucleotide 966 of the coding sequence of the HvLDI gene (SEQ ID NO:2); and
   ii. a mutation of nucleotide C to T at the position corresponding to nucleotide 967 of the coding sequence of the HvLDI gene (SEQ ID NO:2); and
   iii. a mutation of nucleotide C to T at the position corresponding to nucleotide 968 of the coding sequence of the HvLDI gene (SEQ ID NO:2); and
   iv. a mutation of G to A at the position corresponding to nucleotide 990 of the coding sequence of the HvLDI gene (SEQ ID NO:2).
26. The barley plant or part thereof according to any one of the preceding items, wherein said barley plant carries a mutations in the HvLDI gene consisting of a mutation of nucleotide C to T at the position corresponding to nucleotide 966 of the coding sequence of the HvLDI gene (SEQ ID NO:2).
27. The barley plant according to any one of the preceding items, wherein the barley plant is the barley plant deposited under accession number NCIMB 43581 with NCIMB or progeny thereof.
28. The barley plant or part thereof according to any one of items 1 to 25, wherein said barley plant carries a mutations in the HvLDI gene consisting of a mutation of nucleotide C to T at the position corresponding to nucleotide 967 of the coding sequence of the HvLDI gene (SEQ ID NO:2).
29. The barley plant or part thereof according to any one of items 1 to 25, wherein said barley plant carries a mutations in the HvLDI gene consisting of a mutation of nucleotide G to A at the position corresponding to nucleotide 990 of the coding sequence of the HvLDI gene (SEQ ID NO:2).
30. The barley plant or part thereof according to any one of items 1 to 24, wherein said barley plant carries a mutations in the HvLDI gene consisting of a consisting of a mutation of nucleotide C to T at the position corresponding to nucleotide 966 and a mutation of nucleotide C to T at the position corresponding to nucleotide 967 of the coding sequence of the HvLDI gene (SEQ ID NO:2).
31. The barley plant or part thereof according to any one of items 1 to 25, wherein said barley plant carries a mutations in the HvLDI gene consisting of a consisting of a mutation of nucleotide C to T at the position corresponding to nucleotide 966 and a mutation of nucleotide C to T at the position corresponding to nucleotide 968 of the coding sequence of the HvLDI gene (SEQ ID NO:2).
32. The barley plant or part thereof according to any one of items 1 to 24, wherein said barley plant carries a mutations in the HvLDI gene consisting of a consisting of a mutation of nucleotide C to T at the position corresponding to nucleotide 967 and a mutation of nucleotide G to A at the position corresponding to nucleotide 990 of the coding sequence of the HvLDI gene (SEQ ID NO:2).
33. The barley plant or part thereof according to any one of items 1 to 24, wherein said barley plant carries a mutations in the HvLDI gene consisting of a consisting of a mutation of nucleotide C to T at the position corresponding to nucleotide 966 and a mutation of nucleotide G to A at the position corresponding to nucleotide 990 of the coding sequence of the HvLDI gene (SEQ ID NO:2).
34. The barley plant according to any one of the preceding items, wherein the barley plant is the barley plant deposited under accession number NCIMB 43582 with NCIMB or progeny thereof.
35. The barley plant according to any one of the preceding items, wherein the grains of said barley plant have a thousand grain weight of at least 45 gram, such as at least 50 gram, such as at least 55 gram.
36. The barley plant according to any one of the preceding items, wherein the grains of said barley plant have a thousand grain weight of at least 80%, such as at least 85%, such as at least 90%, such as at least 95% compared to grains of a barley plant carrying a HvLDI gene encoding a wt HvLDI polypeptide, but otherwise of the same genotype, when grown under the same conditions.
37. The barley plant according to any one of the preceding items, wherein the grains of said barley plant have a starch content of at least 50%, such as at least 55%, such as at least 60%.
38. The barley plant according to any one of the preceding items, wherein the grains of said barley plant have a starch content of at least 80%, such as at least 85%, such as at least 90%, such as at least 95% compared to grains of a barley plant carrying a HvLDI gene encoding a wt HvLDI polypeptide, but otherwise of the same genotype, when grown under the same conditions.
39. The barley plant according to any one of the preceding items, wherein the barley plant further comprises a mutation in one or more additional genes, for example one or more of the following mutations:
   a. a mutation in the gene encoding LOX-1 resulting in a total loss of functional LOX-1;
   b. a mutation in the gene encoding LOX-2 resulting in a total loss of functional LOX-2
   c. a mutation in the gene encoding MMT resulting in a total loss of functional MMT
   d. a mutation in the gene encoding CsIF6, wherein said mutant gene encodes mutant CsIF6 protein with reduced CsIF6 activity;
   e. a mutation in the gene encoding HRT gene leading to a loss of HRT function;
   f. a mutation in the gene encoding HBL12 gene leading to a loss of HBL function;
   g. a mutation in the gene encoding WRKY38 gene leading to a loss of WRKY38 function;
   h. an ant mutation, for example a mutation in the Hvmyb10 gene.
40. A plant product comprising the barley plant or a part thereof according to any one of the preceding items.
41. The plant product according item 40, wherein the plant product is selected from the group consisting of
   a. malt prepared from grains of said barley plant;
   b. aqueous extract, for example wort, prepared from grains of said barley plant and/or from malt comprising processed grain(s) of said barley plant; and
   c. a beverage, for example beer, prepared from said barley plant of parts thereof.
42. A method of preparing malt, said method comprising the steps of
   a. providing grains of a barley plant according to any one of items 1 to 38;
   b. steeping and germinating said grains under predetermined conditions;
   c. optionally, drying said germinated grains.
43. The method according to item 42, wherein the steeping and germination comprises the following steps:
   a. incubating grains of a barley plant according to any one of items 1 to 38 in an aqueous solution for a period of 5 to 10 h under aeration with a gas comprising oxygen (e.g. pure oxygen or air);
   b. draining off the aqueous solution and subjecting the grains to an air rest for 8 to 16 h, preferably under aeration;
   c. incubating the grains in an aqueous solution for 2 to 10 h under aeration with a gas comprising oxygen; and
   d. draining off the aqueous solution and subjecting the grains to a second air rest phase for 8 to 20 h, preferably under aeration.
   wherein the water content of the grains is at least 20% at any time point after step a. and step c) of item 42 is not performed.
44. The method according to item 42 or 43, wherein said steeping and germination step is performed for at the most 4 days, such as for the most 3 days, preferably for in the range of 48 and 72 hours.
45. A method of preparing an aqueous extract, said method comprising the steps of
   a. providing grains of a barley plant according to any one of items 1 to 39 and/or malt produced according to the method of any one of items 42 to 44;
   b. preparing an aqueous extract of said grains and/or said malt, for example a wort.
46. The method according to item 45, wherein said malt has maintained a water content of at least 20% from it was produced until usage for the preparation of the aqueous extract.
47. The method according to item 45, wherein said aqueous extract have at least 5% more total fermentable sugars compared to an aqueous extract of barley plants carrying a HvLDI gene encoding a wt HvLDI polypeptide, but otherwise of the same genotype, when prepared under the same conditions.
48. The method according to item 45 to 46, wherein said aqueous extract have at least 10% more glucose, fructose and/or maltotriose compared to an aqueous extract of barley plants carrying a HvLDI gene encoding a wt HvLDI polypeptide, but otherwise of the same genotype, when prepared under the same conditions.
49. A method of producing a beverage, said method comprising the steps of
   a. providing grains of a barley plant according to any one of items 1 to 39 and/or malt produced according to the method of any one of items 42 to 44; and
   b. preparing an aqueous extract from said grains and/or malt; or
   c. processing said aqueous extract into a beverage.
50. The method according to item 49, wherein step b. is performed by the method according to of any one of items 45 to 48.
51. A method of preparing a barley plant, the method comprising the steps of
   a. providing barley grains; and
   b. randomly mutagenizing said barley grains,
   c. Selecting barley grains or parts thereof carrying a mutated HvLDI gene encoding a mutant HvLDI polypeptide carrying one of the following mutations
      i. a missense mutation resulting in a change from a proline to a different amino acid in one or more loop regions of HvLDI, wherein the loop regions are selected from the group consisting of amino acids corresponding to position 25 to 44 and amino acids corresponding to position 56 to 62 and amino acids corresponding to position 77 to 78 and amino acids corresponding to position 91 to 111 and amino acids corresponding to position 124 to 147 of SEQ ID NO:1; or
      ii. a missense mutation resulting in a change from a negatively charged amino acid to a non-negatively charged amino acid in one or more alpha helix regions of wt HvLDI, wherein the alpha helix regions are selected from the group consisting of amino acids corresponding to position 45 to 55 and amino acids corresponding to position 63 to 76 and amino acids corresponding to position 79 to 90 and amino acids corresponding to position 112 to 123 of SEQ ID NO:1.

### Sequences

Amino acid sequence of wt HvLDI of Hordeum vulgare.
UniProt accession number Q2V8X0.
SEQ ID NO: 1
MASDHRRFVLSGAVLLSVLAVAAATLESVKDECQPGVDFPHNPLATCHTYVIKRVCGRGPSRPMLVKERCCRELAAV
PDHCRCEALRILMDGVRTPEGRVVEGRLGDRRDCPREEQRAFAATLVTAAECNLSSVQEPGVRLVLLADG
DNA sequence of wt HvLDI of Hordeum vulgare complete CDS DQ285564.1
SEQ ID NO: 2
TTATTGGACACCAAATGTATCATAAACTTGTTTTTTCACCGACAAAATATTGCTCCTCCATTTCGCATTAAAATTGT
CAAGCATGCTTGCAACAGTAACACGAACATTCATAAAAAAAATATTTTTTAAGAAAACATTTACTATTTTTTTGTTA
CTATTCATCTGGGAGCATGTGCTTCCGGAAGCCAAAATGCCCCTTCCAATATGCCCCGTGTAAAAGAAACCCCTTCT
TTCCTAAAAATATATATCATCGTCCGTCATGATACGTTTATGTATTCAACGAAAAATATTTTCGCATGTCACCAAAA
ATGTTTTATATTACACAAGTGAACAAATATGATAAACTCCCTCGTGTTAACTATTTTTTCTGTGAAATAAAAGGATG
ACAATCAAAACAAAAATGTAGACTGTAAACAAAGAAAACATTATTTCCTAGAAATAAAAAAAAAGATTAGAGGGATA
TGTATTGTCGAAACACATGAGGACTAGAACAAAAGAAAAAGGGAAATGAGAAGGAAAAAAGGGGTAACCATTACCCA
AAGAAAACAGAAAGTAAACTAGACGTGTCGAAGGGAAACGGAGTTTGCAGGGGCGTTCCAAATTCAGTTGCAAGAAC
CTCCAAATAAACGCCAACAAGAAAGAAATGAGCATTACTTGCGCGCTTTGCACTCTTATCTCTAGCATCTCCCGATA
CATACATACATGTAGCCTAGCTGCAGATCTTGAATAGCTATTCTTGCCCACCAGGCCAAGAGATTGAACCAACGACC
AATAAACTAGTATCAACAATGGCATCCGACCATCGTCGCTTCGTCCTCTCCGGCGCCGTCTTGCTCTCGGTCCTCGC
CGTCGCCGCCGCCACCCTGGAGAGCGTCAAGGACGAGTGCCAACCAGGGGTGGACTTCCCGCATAACCCGTTAGCCA
CCTGCCACACCTACGTGATAAAACGGGTCTGCGGCCGCGGTCCCAGCCGGCCCATGCTGGTGAAGGAGCGGTGCTGC
CGGGAGCTGGCGGCCGTCCCGGATCACTGCCGGTGCGAGGCGCTGCGCATCCTCATGGACGGGGTGCGCACGCCGGA
GGGCCGCGTGGTTGAGGGACGGCTCGGTGACAGGCGTGACTGCCCGAGGGAGGAGCAGAGGGCGTTCGCCGCCACGC
TTGTCACGGCGGCGGAGTGCAACCTATCGTCCGTCCAGGAGCCGGGAGTACGCTTGGTGCTACTGGCAGATGGATGA
CGATCGAAATGCGCCAAGGTAATGAAGCGGAGTACTGTATACAGAATAAAAGTA

The ATG indicated in bold, represents the start codon for encoding SEQ ID NO: 1. The underlined codons correspond to the codons which are mutated according to the present invention

Amino acid sequence of mutant P60S HvLDI of Hordeum vulgare (HENZ-16a)
SEQ ID NO: 3
MASDHRRFVLSGAVLLSVLAVAAATLESVKDECQPGVDFPHNPLATCHTYVIKRVCGRGSSRPMLVKERCCRELAAV
PDHCRCEALRILMDGVRTPEGRWEGRLGDRRDCPREEQRAFAATLVTAAECNLSSVQEPGVRLVLLADG
Amino acid sequence of mutant P60L HvLDI of Hordeum vulgare (HENZ-16b)
SEQ ID NO: 4
MASDHRRFVLSGAVLLSVLAVAAATLESVKDECQPGVDFPHNPLATCHTYVIKRVCGRGLSRPMLVKERCCRELAAV
PDHCRCEALRILMDGVRTPEGRVVEGRLGDRRDCPREEQRAFAATLVTAAECNLSSVQEPGVRLVLLADG
Amino acid sequence of mutant V66M HvLDI of Hordeum vulgare (HENZ-18)
SEQ ID NO: 5
MASDHRRFVLSGAVLLSVLAVAAATLESVKDECQPGVDFPHNPLATCHTYVIKRVCGRGPSRPMLMKERCCRELAAV
PDHCRCEALRILMDGVRTPEGRVVEGRLGDRRDCPREEQRAFAATLVTAAECNLSSVQEPGVRLVLLADG
Amino acid sequence of mutant E68K HvLDI of Hordeum vulgare (HENZ-31)
SEQ ID NO: 6
MASDHRRFVLSGAVLLSVLAVAAATLESVKDECQPGVDFPHNPLATCHTYVIKRVCGRGPSRPMLVKKRCCRELAAV
PDHCRCEALRILMDGVRTPEGRVVEGRLGDRRDCPREEQRAFAATLVTAAECNLSSVQEPGVRLVLLADG
Amino acid sequence of wt HvLD of Hordeum vulgare.
UniProt accession number Q9FYY0
SEQ ID NO: 7
MAVGETGASVSAAEAEAEATQAFMPDARAYWVTSDLIAWNVGELEAQSVCLYASRAAAMSLSPSNGGIQGYDSKVEL
QPESAGLPETVTQKFPFISSYRAFKVPSSVDVASLVKCQLVVASFGADGKHVDVTGLQLPGVLDDMFAYTGPLGAVF
SEDSVSLHLWAPTAQGVSVCFFDGPAGPALETVQLKESNGVWSVTGPREWENRYYLYEVDVYHPTKAQVLKCLAGDP
YTRSLSANGARTWLVDINNETLKPASWDELADEKPKLDSFSDITIYELHIRDFSAHDGTVDSDSRGGFRAFAYQASA
GMEHLRKLSDAGLTHVHLLPSFHFAGVDDIKSNWKFVDECELATFPPGSDMQQAAVVAIQEEDPYNWGYNPVLWGVP
KGSYASDPDGPSRIIEYRQMVQALNRIGLCVVMDVVYNHLDSSGPCGISSVLDKIVPGYYVRRDTNGQIENSAAMNN
TASEHFMVDRLIVDDLLNWAVNYKVDGFRFDLMGHIMKRTMMRAKSALQSLTTDAHGVDGSKIYLYGEGWDFAEVAR
NQRGINGSQLNMSGTGIGSFNDRIRDAINGGNPFGNPLQQGFNTGLFLEPNGFYQGNEADTRRSLATYADQIQIGLA
GNLRDYVLISHTGEAKKGSEIHTFDGLPVGYTASPIETINYVSAHDNETLFDVISVKTPMILSVDERCRINHLASSM
MALSQGIPFFHAGDEILRSKSIDRDSYNSGDWFNKLDFTYETNNWGVGLPPSEKNEDNWPLMKPRLENPSFKPAKGH
ILAALDSFVDILKIRYSSPLFRLSTANDIKQRVRFHNTGPSLVPGVIVMGIEDARGESPEMAQLDTNFSYVVTVFNV
CPHEVSMDIPALASMGFELHPVQVNSSDTLVRKSAYEAATGRFTVPGRTVSVFVEPRC
Nucleotide sequence of wt HvLD of Hordeum vulgare.
GenBank accession number AF252635.1
SEQ ID NO: 8
ATGGCGGTCGGGGAGACCGGCGCCTCCGTCTCCGCAGCCGAGGCCGAGGCCGAGGCCACCCAGGCGTTCATGCCGGA
CGCCAGGGCGTACTGGGTGACGAGCGACCTCATCGCCTGGAACGTCGGCGAGCTGGAAGCGCAGTCCGTCTGCCTGT
ACGCCAGCAGAGCCGCCGCGATGAGCCTCTCGCCGTCGAATGGCGGCATCCAAGGCTACGACTCCAAGGTTGAGCTG
CAACCGGAGAGCGCCGGGCTCCCGGAAACCGTGACCCAGAAGTTCCCTTTCATCAGCAGTTACAGAGCATTCAAGGT
CCCGAGCTCTGTCGACGTCGCCAGCCTTGTGAAATGCCAACTGGTCGTCGCTTCTTTCGGCGCTGACGGGAAACACG
TAGATGTTACTGGACTGCAATTACCCGGCGTGCTGGATGATATGTTCGCATACACGGGACCGCTCGGTGCGGTTTTC
AGCGAGGACTCTGTGAGCCTGCACCTTTGGGCTCCTACAGCACAGGGCGTGAGCGTGTGCTTCTTTGATGGTCCAGC
AGGCCCTGCGCTAGAGACGGTGCAGCTCAAGGAGTCAAATGGTGTTTGGAGTGTCACTGGACCAAGAGAGTGGGAAA
ACCGGTACTATTTGTATGAAGTCGACGTGTATCATCCAACTAAGGCGCAGGTTCTGAAATGTTTAGCTGGTGACCCT
TATACTAGAAGCCTTTCTGCAAATGGAGCGCGTACCTGGTTGGTTGACATTAACAATGAGACATTGAAGCCGGCTTC
CTGGGATGAATTGGCTGATGAGAAGCCAAAACTTGATTCCTTCTCTGACATAACCATCTATGAATTGCACATTCGTG
ATTTTAGCGCCCACGATGGCACAGTGGACAGTGACTCTCGTGGAGGATTTCGTGCATTTGCATATCAGGCCTCGGCA
GGAATGGAGCACCTACGGAAATTATCTGATGCTGGTTTGACTCATGTGCATTTGTTGCCAAGCTTTCATTTTGCTGG
CGTTGACGACATTAAGAGCAACTGGAAATTTGTCGATGAGTGTGAACTAGCAACATTCCCTCCAGGGTCAGATATGC
AACAAGCAGCAGTAGTAGCTATTCAGGAAGAGGACCCTTATAATTGGGGGTATAACCCTGTGCTCTGGGGGGTTCCA
AAAGGAAGCTATGCAAGTGACCCTGATGGCCCGAGTCGAATTATTGAATATCGTCAGATGGTCCAGGCCCTCAATCG
CATAGGTCTTTGTGTTGTCATGGATGTTGTATACAATCATCTAGACTCAAGTGGCCCCTGCGGTATCAGCTCAGTGC
TTGACAAGATTGTTCCTGGGTACTATGTTAGAAGGGATACTAATGGCCAGATTGAGAACAGTGCAGCTATGAACAAT
ACAGCAAGTGAGCATTTCATGGTTGATAGGTTAATCGTGGATGACCTTTTGAACTGGGCAGTAAACTACAAAGTTGA
CGGGTTCAGATTTGATCTTATGGGCCATATCATGAAACGCACAATGATGAGAGCAAAATCTGCTCTTCAAAGCCTTA
CAACAGATGCACATGGAGTTGATGGTTCAAAAATATACTTGTATGGTGAAGGATGGGACTTCGCTGAAGTTGCACGC
AATCAACGTGGAATAAATGGGTCCCAGCTTAATATGAGTGGAACGGGGATTGGTAGCTTCAATGATAGAATCCGGGA
TGCTATTAATGGGGGTAATCCCTTTGGTAATCCGCTCCAGCAAGGCTTCAATACTGGTCTGTTCTTAGAGCCGAATG
GGTTTTATCAGGGCAATGAAGCAGATACCAGGCGCTCGCTCGCTACTTATGCTGACCAAATACAGATTGGACTAGCT
GGTAATCTGAGGGATTATGTACTAATATCTCATACTGGAGAAGCTAAGAAGGGATCAGAAATTCACACTTTTGATGG
ATTACCAGTAGGCTATACTGCGTCCCCAATAGAAACGATAAACTATGTTTCTGCTCATGACAATGAGACTCTATTTG
ATGTTATCAGTGTGAAGACCCCAATGATCCTTTCAGTTGATGAGAGATGCAGGATAAATCATTTGGCCTCCAGCATG
ATGGCATTATCCCAGGGAATACCCTTCTTCCACGCTGGTGACGAGATACTAAGATCTAAGTCCATCGACCGAGATTC
ATATAACTCTGGTGATTGGTTTAACAAGCTTGATTTTACCTATGAAACAAACAATTGGGGTGTTGGGCTTCCTCCAA
GTGAAAAGAACGAAGATAATTGGCCCCTGATGAAACCAAGATTGGAAAATCCGTCTTTTAAACCTGCAAAAGGACAC
ATTCTTGCTGCCCTAGACAGTTTTGTTGACATCTTGAAGATCAGATACTCATCTCCACTTTTTCGTCTCAGTACAGC
AAATGACATTAAGCAAAGGGTACGCTTTCACAACACAGGGCCCTCCTTAGTCCCAGGTGTTATTGTCATGGGCATTG
AAGATGCACGAGGTGAGAGCCCCGAGATGGCTCAATTAGACACGAACTTCTCTTATGTCGTAACCGTCTTCAATGTG
TGTCCGCACGAAGTGTCCATGGATATCCCCGCTCTCGCTTCGATGGGGTTTGAACTGCATCCTGTGCAGGTGAATTC
ATCAGATACTTTGGTGAGGAAATCGGCGTACGAGGCCGCGACGGGCAGGTTCACCGTGCCCGGAAGAACCGTGTCAG
TCTTTGTCGAACCTCGGTGTTGA

### REFERENCES

Blennow, A., et al. (1998) The degree of starch phosphorylation is related to the chain length distribution of the neutral and the phosphorylated chains of amylopectin. Carbohydr. Res. 307: 45-54.

Calum et al 2004 J Inst Brewing 110(4): 284-296

Himi et al., 2012 Euphytica (2012) 188:141-151 DOI 10.1007/s10681-011-0552-5

Hough J. S., Briggs D. E., Stevens R. and Young T. W., Malting and Brewing Science, Springer US, 1982, doi 10.1007/978-1-4615-1799-3

Huang et al. BMC Plant Biol. 2014; 14: 117. PMID: 24885294.

Jende-Strid, 1993, Hereditas 119:187-204

Li et al. (2015 Apr. 6) Nucleic Acids Research 43 (W1) :W580-4 PMID: 25845596

McWilliam et al., (2013 May 13) Nucleic Acids Research 41 (Web Server issue): W597-600 PMID: 23671338

Møller et al. (2015). The journal of biological chemistry vil.290, no 20, pp 12614-12629.

Shaik, S. S., et al. (2014) Starch bioengineering affects cereal grain germination and seedling establishment. J. Exp. Bot. 65: 2257-2270.

Sievers et al. (2011 Oct. 11) Molecular Systems Biology 7:539, PMID: 21988835

Stahl et al 2007 Plant Science 172(3): 452-561

### EXAMPLES

### Example 1. Screening for Barley Mutants (Hv) with Specific Mutations in the Gene for LDI

First, four HvLDI with a specific mutation, leading to the substitution of an amino acid residue in LDI, were prepared (Table 2).

TABLE 2
HvLDI mutants.

| | Nucleotide change in | Amino acid change |
|---|---|---|
| HvLDI mutant | coding sequence | in protein |
| (Internal ID) | (SEQ ID NO: 2) | (SEQ ID NO: 1) |
| HENZ-16a | C→T (966) | Pro→Ser (60) |
| HENZ-16b | C→T (967) | Pro→Leu (60) |
| HENZ-18 | G→A (984) | Val→Met (66) |
| HENZ-31 | G→A (990) | Glu→Lys (68) |

Next, HENZ-16a, HENZ-18, HENZ-31 barley plant mutants were prepared by random mutagenesis following by identification by a ddPCR based method performed essentially as described in international patent application WO 2018/001884. More specifically, a pool of randomly mutagenized barley grains (parent variety Paustian and Planet) was prepared, followed by preparation of an ordered library as described in international patent application WO 2018/001884 in WS1 and WS2 on p. 66-69 as well as in Examples 1 to 2 (hereby incorporated by reference).

The wild type HvLDI gene encoding HvLDI of SEQ ID NO:1 (UniProt accession number Q2V8X0) was used as a reference gene for wild type HvLDI.

The HENZ-16a, HENZ-16b, HENZ-18, HENZ-31 barley plant mutants were identified and selected as described in international patent application WO 2018/001884 in WS3 and WS4 on p. 67-72 as well as in Examples 3 to 15 using the primers and probes specified in Table 3 below.

The background, i.e. the parent plant, of HENZ-16a, HENZ-16b and HENZ-18 is Paustian, and the background of HENZ-31 is Planet. Paustian and Planet barley plants contain a wild type HvLDI gene encoding HvLDI polypetide. Paustian is available from Sejet Plant Breeding, Nørremarksvej 67, 8700 Horsens DK. Planet is available from RAGT Semences, Rue Emile Singla, 12000 Rodez, France.

TABLE 3
Primers and probes designed for the specific barley plant mutants

| | | | Mutant-specific | Reference- |
|---|---|---|---|---|
| | Target- | Target- | detection probe | specific detection |
| Barley | specific | specific | labelled with | probe labelled |
| plant | forward | reverse | 6-carboxy- | with hexachloro- |
| mutant | primer | primer | fluorescein (FAM) | fluorescein (HEX) |
| HENZ-16a | CTACGTGAT | CCGCTCCTT | CGGCTGGAACC | CCGGCTGGGA |
| | AAAACGGGT | CACCAG | GC (SEQ ID | CCG (SEQ ID |
| | C (SEQ ID | (SEQ ID | NO: 11) | NO: 12) |
| | NO: 9) | NO: 10) | | |
| HENZ-18 | GCCGCGGTC | CAGTGATCC | ATGCTGATGAA | ATGCTGGTGAA |
| | CCA | GGGACG | GGAGCG (SEQ | GGAGC (SEQ ID |
| | (SEQ ID | (SEQ ID | ID NO: 15) | NO: 16) |
| | NO: 13) | NO: 14) | | |
| HENZ-31 | GCCGCGGTC | CAGTGATCC | TGGTGAAGAAG | TGGTGAAGGAG |
| | CCA (SEQ ID | GGGACG (SEQ | CGGTG (SEQ | CGGT (SEQ ID |
| | NO: 17) | ID NO: 18) | ID NO: 19) | NO: 20 |

### Example 2. In Vitro Binding Studies

### Method

A commercially available in vitro assay (Pullulanase/Limit-Dextrinase Assay Kit PullG6 Method, Megazyme, Ireland) was used according to manufacturer's instructions to assess the ability of recombinant expressed HvLDI polypeptide to inhibit the activity of recombinant Hordeum vulgare limit dextrinase (HvLD).

The PullG6 method for the measurement of limit dextrinase activity is based on a water soluble defined substrate, namely 4,6-O-benzylidene-4-nitrophenyl-6³-α-D-maltotriosyl-maltotriose (BPNPG3G3), coupled with the ancillary enzymes α-glucosidase and β-glucosidase.

The specific hydrolysis of the 1,6-α-linkage in the substrate by limit-dextrinase is followed by further hydrolysis to glucose and 4-nitrophenol by the α-glucosidase and β-glucosidase enzymes and the reaction terminated by addition of alkaline solution. The absorbance at 400 nm can be directly correlated to limit dextrinase activity.

For synthesis of high levels of soluble, recombinant HvLDI, the corresponding gene (NS03) was inserted into the E. coli expression vector pSol-SUMO (Lucigen, USA).

Chemically competent E. cloni 10G cells transformed with wild type HvLDI or containing nucleotide mutations in pSol-SUMO were used for heterologous expression. Protein purification was achieved using a 5-mL immobilised metal-affinity chromatography (IMAC) crude column (GE Healthcare, USA) and N-terminal tag was removed by TEV protease cleavage.

The protein concentration of enriched, cleaved and concentrated HvLDI was determined using the Pierce 660 nm Protein Assay (ThermoFisher Scientific, USA) before in vitro inhibition assay.

In the case of recombinant wt HvLD, the corresponding gene (SEQ ID NO:7) was inserted into the pET28a expression vector (Novagen, USA) (Now Merck biosciences, Germany). Expression was carried out in BL21(DE3) expression cells (New England Biolabs, USA). The protein was purified in a HisTrap FF metal affinity chromatography column (GE Heathcare, USA), exchanges into PBS buffer and concentrated to 4.6 mg/mL prior to its utilization in the assays.

The assay was down-scaled in order to be carried out in a total reaction volume of 25 μL. In preliminary experiments a final concentration of 1.5 μM HvLD resulted in a good signal under standard reaction conditions with the ability to detect inhibition but also activation, if necessary.

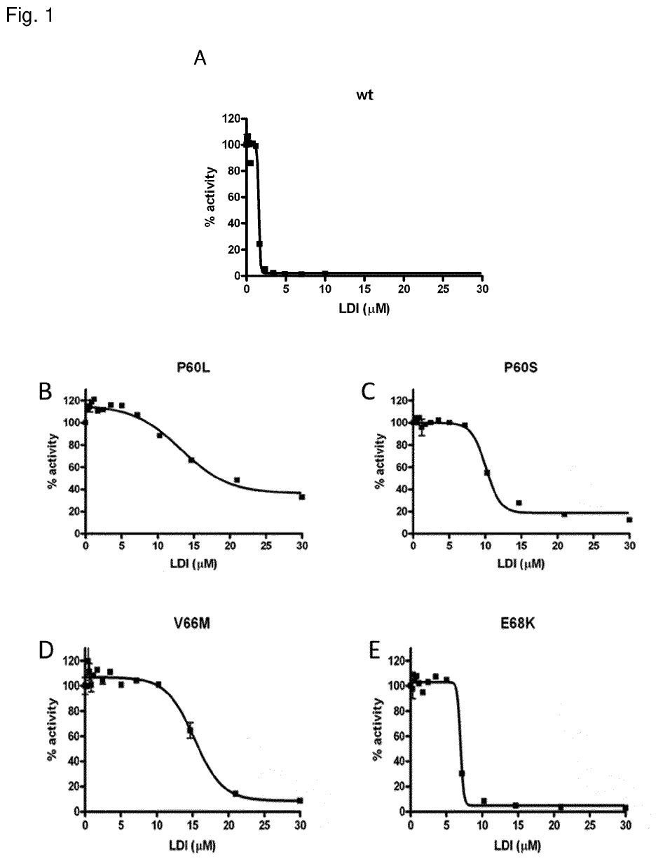

A serial dilution of purified, recombinant mutant or wt HvLDI was made in reaction buffer (100 mM sodium maleate, pH 5.5) and 10 μL of each dilution were mixed with 10 μL of 6 μM recombinant HvLD in the same buffer. The mix was incubated for 5 min at room temperature. The reaction was started by the addition of 12.5 μL HvLD/HvLDI to the same volume of P6 reagent. The reaction proceeded for 30 min at 40° C. and was stopped by the addition of 187.5 μL stopping reagent [2% (w/v) Tris-base solution, pH 9.0]. An aliquot of 50 μL of each stopped reaction was transferred to individual wells on a half-area, flat-bottom 96-well microplate. A400 nm was measured from the bottom on a SpectraMax 340PC384 microplate reader (Molecular Devices, USA), using path correction to account for minor differences in volume. The data was exported, the background (HvLD was replaced by the same volume of reaction buffer) was subtracted and the absorbance was plotted against the concentration of HvLDI in the assay. The half maximal inhibitory concentration (IC₅₀) was determined using GraphPad Prism (version 4, GraphPad Software, USA). See FIG. 1 for free HvLD activity.

### Results

Purified recombinant wt and mutant HvLDI were used in a commercially available enzyme assay to detect free HvLD activity. The potency of wt HvLDI and mutant HvLDI to inhibit recombinant expressed HvLD was assessed by the amount of chromophore released during the assay. For all mutants tested a noticeable higher concentration was necessary to see an inhibitory effect compared to wt (see FIG. 1 and Table 4).

The mutations P60, V66M and E68K showed a considerable reduction in the ability to inhibit HvLD. HvLDI-P60L does not fully inhibit HvLD even at the highest concentration tested. Considerably higher concentrations of HvLDI-P60L, HvLDI-P60S, HvLDI-V66M and HvLDI E68K are necessary to achieve inhibition of HvLD % activity compared to wt-LDI barley plants.

TABLE 4

| LDI* | IC₅₀ (μM) | Standard deviation |
|---|---|---|
| WT | 1.56 | 0.05 |
| P60L | 13.2 | 0.6 |
| P60S | 10.1 | 0.2 |
| V66M | 15.3 | 0.5 |
| E68K | 7.10 | n/a** |

*WT is LDI of SEQ ID NO: 1, mutations in relation to SEQ ID NO: 1 indicated
**Standard deviation not calculated, but visual inspection of graphs showed that data is useful

### Example 3. Germination Test

The following barley plants HENZ-16, HENZ-18, HENZ-31, Paustian and Planet were grown on the field in New Zealand season 2017/2018 under standard conditions and harvested once grains had reached maturity.

All barley grain samples were evaluated for the parameters germination index (G index), germination energy and water sensitivity. Data are based on two sample sizes of 100 grains for a 4 mL germination test and a sample size of 100 barley grains for an 8 mL germination test according to Analytical-EBC Method 3.6.2 Germinative Energy of Barley: BRF Method, 2004.

The germinating grains were counted after 24, 48 and 72 hours incubation on a Petri dish with two filter papers (Whatman, Grade 1, 85 mm, CAT No. 1001-085) and 4 ml of milli-Q water in a humidified box at 20° C.

### G Index

The G index is an indicator of the germination through a period of 3 days, described through the following equation: 10*(x+y+z)/(x+2*y+3*z), wherein x is number of germinating grains counted at 24 hr, y is the number of germinating grains counted at 48 hr and z is the number of germinating grains counted at 72 hr

### Germination Energy

The germination energy describes the percentage of germinated grains of the total grains in the germination test. The germination energy is calculated on data based on a count of germinated grains every 24 hour for 3 days.

### Water Sensitivity

Water sensitivity is measured by counting germinated grains after 72 hours incubation on a Petri dish with 8 ml of milli-Q water and comparing the 4 mL: G Energy_{4 ml}−G Energy_{8 ml}.

### Results

TABLE 5

| | | G Energy, 4 ml | G Energy, 8 ml | Water Sensitivity |
|---|---|---|---|---|
| | G Index | (%) | (%) | (%) |
| HENZ-16 | 8.3 | 96.5 | 44.0 | 52.5 |
| HENZ-18 | 7.3 | 96.0 | 51.0 | 45.0 |
| Paustian | 8.5 | 96.5 | 38.0 | 58.5 |
| HENZ-31 | 8.0 | 98.0 | 66.0 | 32.0 |
| Planet | 8.0 | 98.5 | 84.0 | 14.5 |

No significant difference in germination index (G index), germination energy and water sensitivity were observed for grains from HvLDI mutant barley plants compared grains from wt barley plants.

### Example 4. Gelatinization Temperature

The following barley plants HENZ-16a, HENZ-31, Paustian and Planet were grown in Denmark 2017 under standard conditions and harvested once grains had reached maturity

Barley grain samples were milled to fine flour using a laboratory Retsch ball mill. Approximately 115 mg of barley flour were mixed with 3 times that weight in water and 25 uL of the suspension were pipetted into aluminium pans in. Pans were sealed hermetically. The gelatinization temperatures were determined by heating the pan in the Differential Scanning calorimeter (DSC-1 STARe System, Mettler-Toledo) from 40 to 90° C. at a heating rate of 10° C./min. An empty pan was used as a reference.

Results:

TABLE 6

| | GT (° C.) |
|---|---|
| HENZ-16a | 61.5 ± 0.1 |
| Paustian | 61.9 ± 0.1 |
| HENZ-31 | 61.5 ± 0.2 |
| Planet | 61.0 ± 0.03 |

No significant difference in gelatinization temperature were observed.

### Example 5. Flex-Malt Malting Process

Wild type grains of cv. Paustian or Planet, and grains of the barley plants carrying a mutation in HvLDI as described in Example 1 were subjected to air accelerated abrasion for one minute using a custom made device to remove about 3-4% of the husk before steeping.

Grains were placed in an aqueous solution in a Plexiglass cylinder and constantly aerated with atmospheric air from beneath the column of grain. Airflow was set using a SmartTrak® 50 mass flow meter and controller (Sierra, Calif., USA) and temperature was measured using a Testo 735 precision thermometer (Testo, Germany).

The wt and mutated barley grains were incubated for 24h in water adjusted to 1 μM gibberellic acid (GA₃, G7645, Sigma-Aldrich), 0.01% Antifoam-204 (Sigma-Aldrich) and 0.01% H₂O₂ (Apoteket, Denmark). Incubation was at 23° C., and the grains were aerated with 90 L/h atmospheric air. After draining, the grains are aerated with 90 L/h atmospheric air for 24 hours.

Gibberellic acid (GA) is a phytohormone that activates the aleurone layer in germinating barley. Many maltsters add GA at low concentration during the malting process. Here, GA were supplemented to the water for incubation of the grains at the start of the process. A GA₃ solution was prepared from gibberellic acid (G7645, Sigma-Aldrich, St. Louis, Mo., USA) in absolute ethanol and added to the water.

During the entire incubation air is lead through the moist cereal grains from the bottom of the tank. In the flex-malting process the grains are not kiln dried but milled and mashed without any drying step.

### Example 6. VLB Malting Process

25 kg barley grains from HENZ-16a and Paustian barley plants were malted at VLB, Berlin. Steeping was performed at 18° C. and germination for 5 days at 14.5° C. Target water content of the grains was 43% before grains were kiln dried.

### Example 7. Methods for Determining Hydrolytic Enzymes Activity

During germination, the barley grain begins to secrete a range of hydrolytic enzymes, such as alpha-amylases, limit dextrinases and (1,3;1,4)-beta-glucanases. Typically, these enzyme activities can be detected in a timely coordinated manner.

### Sample Preparation

72 h germinated grain samples from HENZ-16, HENZ-18, HENZ-31, Paustian and Planet were prepared by germinating 100 grains for 72 hours on a Petri dish with two filter papers (Whatman, Grade 1, 85mm, CAT No. 1001-085) and 4 ml of milli-Q water in a humidified box at 20° C. EBC19 malt prepared according to European Brewing Congress standard EBC19 was included as a control in the experiment.

Flex-malted grain samples from HENZ-16a, HENZ-31, Paustian and Planet were prepared according to the method described in Example 5.

VLB malted grain samples from HENZ-16a and Paustian were prepared according to the method described in Example 6.

All the 72 h germinated grain and Flex-malted samples were frozen and dried by evaporating water in a freeze dryer (ScanVac CoolSafe 4L, LaboGene) for 72 hours.

Prior to enzyme activity analysis all the samples are milled using a standard Cyclotech mill (FOSS, Denmark) to generate flour. All measurements of enzyme activity in germinated barley grains were made within 48 h after milling of the sample.

### Alpha-Amylase Activity

The α-amylase activity was determined according to a downscaled version of the Ceralpha method from Megazyme, Ireland (K-CERA), starting from 250 mg of flour.

### Results

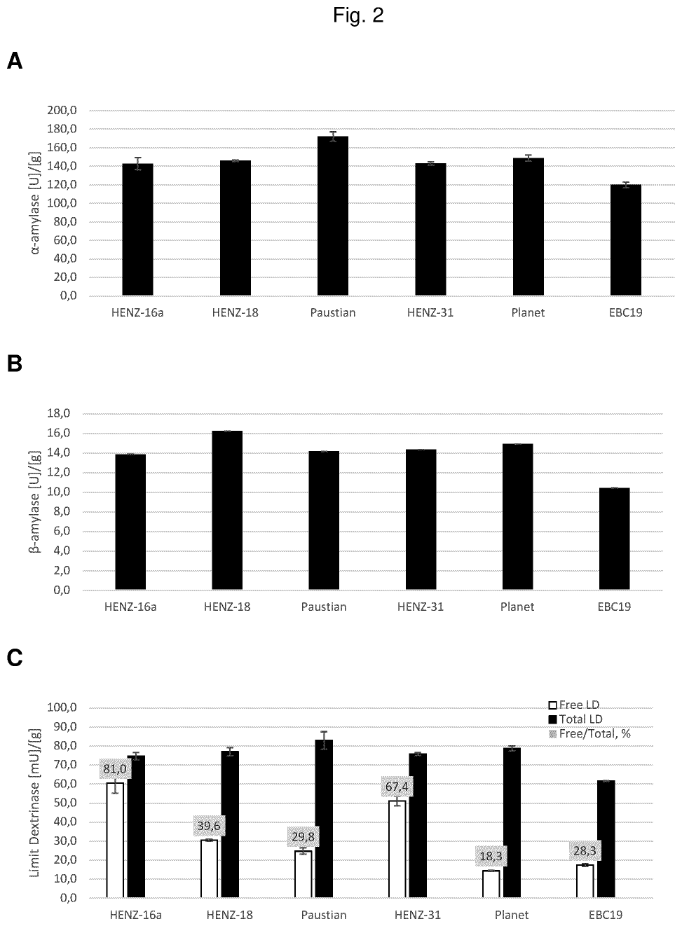

Comparable alpha-amylase activity [U]/[g] was found in germinating grains, flex-malted grains and VLB malted grains from HvLDI barley plant mutants and control barley plants. See FIGS. 2A, 3A and 4A.

β-Amylase Activity

The beta-amylase activity was determined according to a downscaled version of the Betamyl-3 method from Megazyme, Ireland (K-BETA3), starting from 250 mg of flour.

### Results

Comparable beta-amylase activity [U]/[g] was found in germinating grains, flex-malt malted grains and VLB malted grains from HvLDI barley plant mutants and control barley plants. See FIGS. 2B, 3B and 4B.

### Free and Total Limit Dextrinase Activity

The Limit Dextrinase activity is determined according to a downscaled version of the PullG6 method from Megazyme (K-PullG6), starting from 250 mg of flour. Free limit dextrinase activity is measured after extraction of the flour into 2.5 ml of 0.1 M maleic acid pH 4.7 for 1 h at 40° C., with regular mixing every 15 minutes, while total limit dextrinase activity is measured after extraction of the flour into 2.5 ml of 0.1 M maleic acid pH 4.7 containing 25 mM dithiothreitol for 1 h at 40° C., with regular mixing every 15 minutes. After extraction the samples are centrifuged for 10 minutes at 10000 rpm in a benchtop centrifuge (Heraeus Pico17 centrifuge, Thermo Scientific™) and the supernatant transferred to a 500 ul sample cup (Thermo Scientific™). The assay is performed using a custom made assay in a Gallery™ Plus Beermaster Discrete Analyzer (Thermo Scientific™). 24 ul of supernatant are incubated with 24 ul of PullG6 substrate and the reaction let to proceed for 1 h at 37° C. The reaction is stopped by addition of 240 ul of Trizma 2% and the absorbance at 400nm is measured after subtraction of the reaction blank according to the PullG6 method from Megazyme (K-PullG6, Megazyme, Ireland).

### Results

### Results Total Limit Dextrinase

72 h germinated grains and flex-malted grains from HvLDI barley plant mutants were found to have comparable total limit dextrinase activity [mU]/[g] compared to control barley plants. See FIGS. 2C and 3C. Total limit dextrinase activity in VLB malted grains was comparable to total limit dextrinase activity in pilsner malted grains. Due to the malting conditions used for the VLB malted grains, total limit dextrinase levels were lower in grains from VLB malted grains compared to standard pilsner malt. See FIG. 4C.

### Results Free and Free/Total Limit Dextrinase

Free limit dextrinase activity, as well at the ratio of Free/Total limit dextrinase were higher in germinating grains from the HENZ-16a and HENZ-31 barley mutants compared to the two control barley plants Paustian and Planet, and HENZ-18 and EBC19 (FIG. 2C).

Free limit dextrinase activity, as well at the ratio of Free/Total limit dextrinase were higher in flex-malted grains from the HENZ-16a and HENZ-31 barley mutants compared to the two control barley plants Paustian and Planet (FIG. 3C).

Free limit dextrinase activity, as well at the ratio of Free/Total limit dextrinase were higher in VLB malted grains from the HENZ-16a barley mutant compared to the control barley plant Paustian as well as Pilsner malt (FIG. 4C).

### Results Kinetic Measurements

Kinetic measurements were additionally performed on flex-malted grains. Substrate kinetics are measured for free limit dextrinase after extraction of the flexmalted flour into 0.1 M maleic acid pH 4.7 for 1 h at 40° C. and for total limit dextrinase after extraction of the flour into 0.1 M maleic acid pH 4.7 containing 25 mM dithiothreitol for 1 h at 40° C. Extractions were performed as for PullG6 method from Megazyme.

50 ul of extract were incubated in assay tubes with 50 ul of PullG6 substrate at a final concentration of 0-3 mM for 30 min at 40° C. Then reaction was stopped with 750 ul Trizma 2% and 400 mm absorbance was measured in a Genesys 10S UV-Vis spectrophotometer (Thermo Scientific™) after subtraction of the reaction blank according to the PullG6 method from Megazyme. (K-PullG6, Megazyme, Ireland). Data were fit with a Michaelis-Menten function, using a public available website, ic50.tk, and Km determined as the concentration of substrate when the reaction reaches half of Vmax.

TABLE 7

| | Free HvLD activity | | Total HvLD activity | |
|---|---|---|---|---|
| | Km (mM) | Vmax | Km (mM) | Vmax |
| HENZ-16 | 0.95 ± 0.12 | 46.9 ± 2.4 | 0.12 ± 0.04 | 69.3 ± 4.1 |
| Paustian | 7.48 ± 3.83 | 52.2 ± 20.9 | 0.11 ± 0.04 | 65.5 ± 3.7 |
| HENZ-31 | 1.06 ± 0.12 | 58.9 ± 2.9 | 0.11 ± 0.03 | 74.6 ± 3.5 |
| Planet | 5.98 ± 0.2.42 | 60.8 ± 18.4 | 0.14 ± 0.04 | 69.7 ± 3.4 |

Km for free HvLD was lower for HENZ-16a and HENZ-31 compared to the two control barley plants, Planet and Paustian, see table 7.

Similar Km for total HvLDI was observed for HENZ-16a, HENZ-31, Planet and Paustian (Table 7).

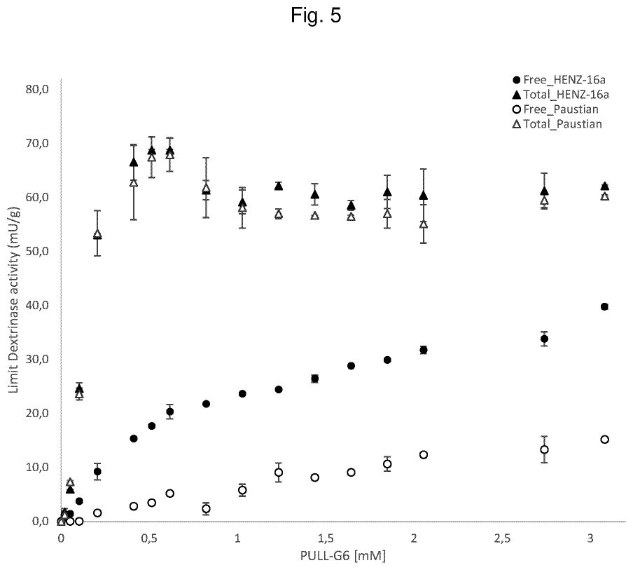

Free limit dextrinase activity was higher in HENZ-16a compared to Paustian. See also FIG. 5.

### Example 8: Mashing and Sugar Analysis in Wort

VLB malt and dry flex-malt were milled to powder using a standard Cyclotech mill (FOSS, Denmark). EBC19 malt prepared according to European Brewing Congress standard EBC19 was included as a control in the experiment.

### Mashing of Dry Flex-Malt

70 g of dry matter were mixed in a water : grist ratio 5:1 and mashed in a Lochner mashing equipment according to the following mashing program: 10 minutes at 52° C., 50 minutes at 65° C. and 5 minutes at 78° C., spaced-out by a temperature ramping of 1 degree/min. This process may also be referred to as “mashing”.

Mashing of VLB Malt

15 g of dry matter were mixed in a water:grist ratio 4:1 and mashed in a mashing robot equipment (Zinsser Analytics, Germany) according to the following mashing program: 15 minutes at 52° C., 45 minutes at 65° C., 15 minutes at 72° C. and 5 minutes at 78° C., spaced-out by a temperature ramping of 1 degree/min. This process may also be referred to as “mashing”.

The levels of fermentable sugars, such as fructose, sucrose, glucose, maltose and maltotriose, as well as other sugars were determined in the wort. The analysis were performed on filtered wort neutralized in 0.1M NaOH. Soluble sugars were determined by High-Performance Anion-Exchange Chromatography Coupled with Pulsed Electrochemical Detection (HPAEC-PAD).

### Sugar Analysis in Wort Prepared from Dry flex-Malt

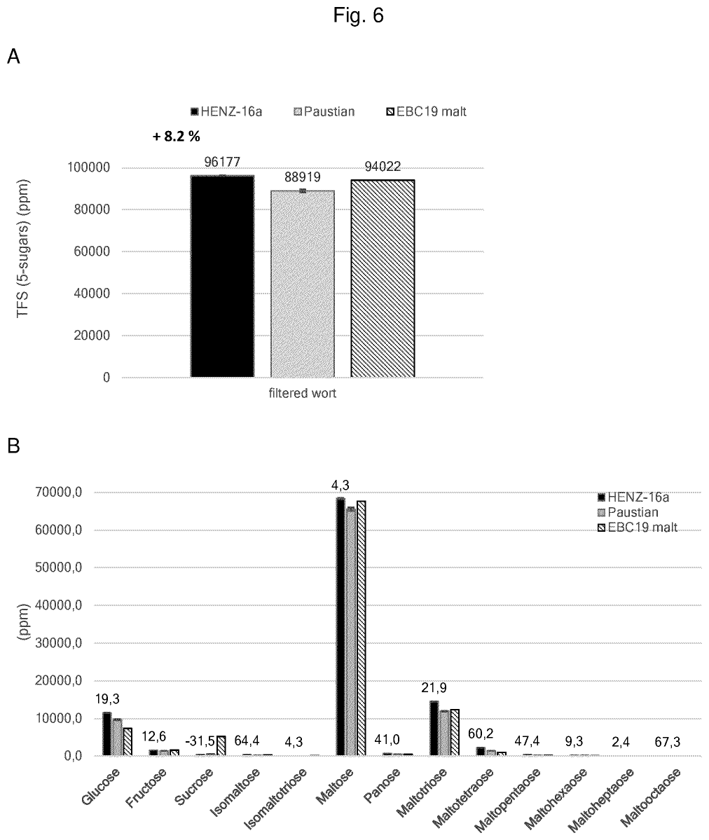

The results are shown in FIG. 6. The results in FIG. 6A demonstrate that wort prepared from grains from the HENZ-16a barley mutant contains 8.2% more fermentable sugars compared to wort prepared form grains from Paustian.

More specifically, the levels of glucose, fructose, isomaltose, isomaltotriose, maltose, panose, maltotriose, maltotetraose, maltopentaose, maltohexaose, maltoheptaose and maltoactaose in wort prepared from HENZ-16a are higher compared to the levels of these sugars in wort prepared from Paustian (FIG. 6B).

### Sugar Analysis in Wort Prepared From Dry VLB Malt

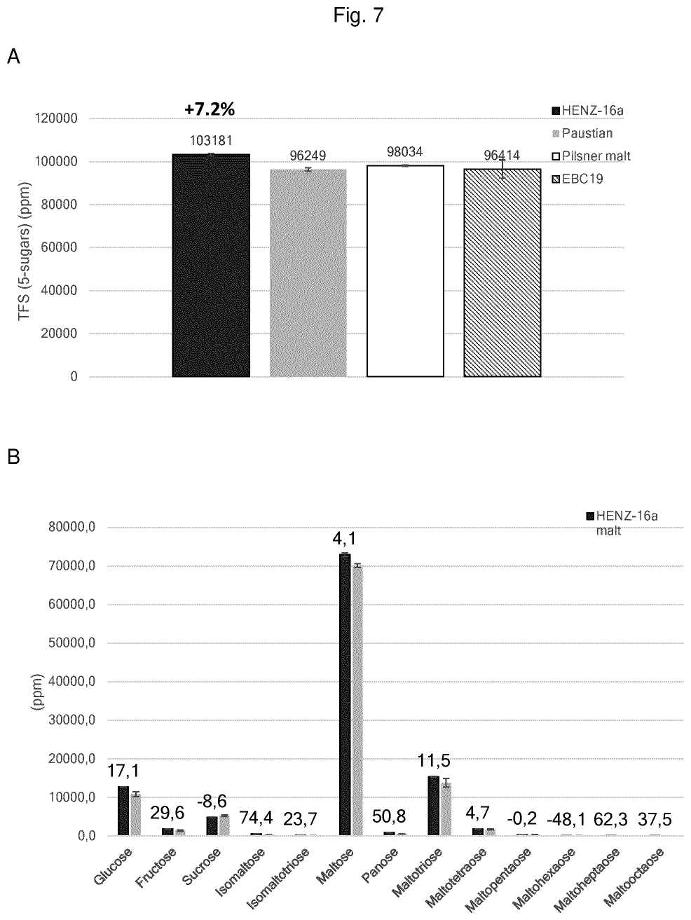

The results are shown in FIG. 7. The results in FIG. 7A demonstrate that wort prepared from grains from the HENZ-16a barley mutant contains 7.2% more fermentable sugars compared to wort prepared from grains from Paustian (FIG. 7A).

More specifically, the levels of glucose, fructose, isomaltose, isomaltotriose, maltose, panose, maltotriose, maltotetraose, maltoheptaose and maltoactaose in wort prepared from HENZ-16a are higher compared to the levels of these sugars in wort prepared from Paustian (FIG. 7B).

### Example 9. Amylopectin Chain Length Distribution Analysis/Degree of Polymerization

### Denmark Season 2017

HENZ-16a, HENZ-18 and HENZ-31, Planet and Paustian barley plants were grown in neighbouring plots in Denmark in the season 2017.

### New Zealand Season 2017/2018

HENZ-16a, HENZ-18 and HENZ-31, Planet and Paustian barley plants were grown in neighbouring plots in New Zealand in the season 2017/18. The harvested grains were analysed as described below.

Starch was isolated from flour (2mg) following Shaik et al. (2014). Starch was debranched with Pseudomonas spearoides isoamylase and Bacillus licheniformis pullulanase (Megazyme, Ireland) and analyzed in an ICS-3000 chromatography system (Dionex) using CarboPac PA100 analytical columns following Blennow et al. (1998).

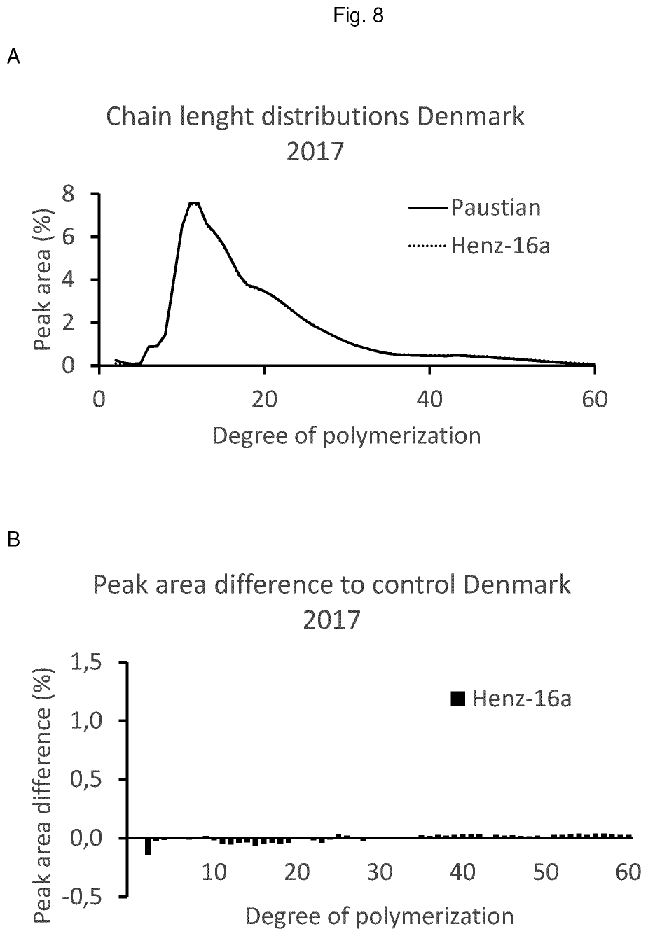

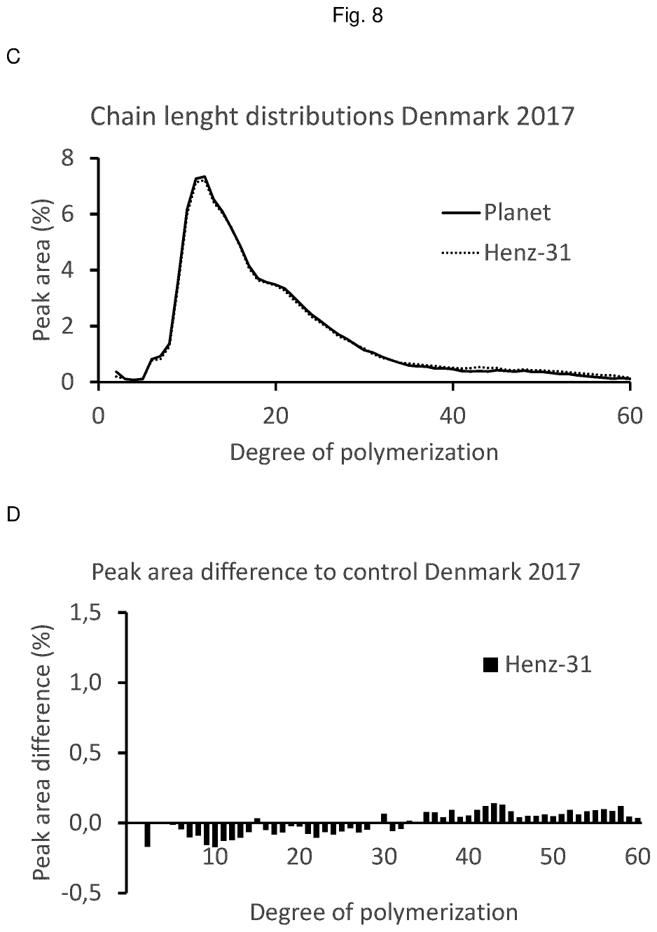

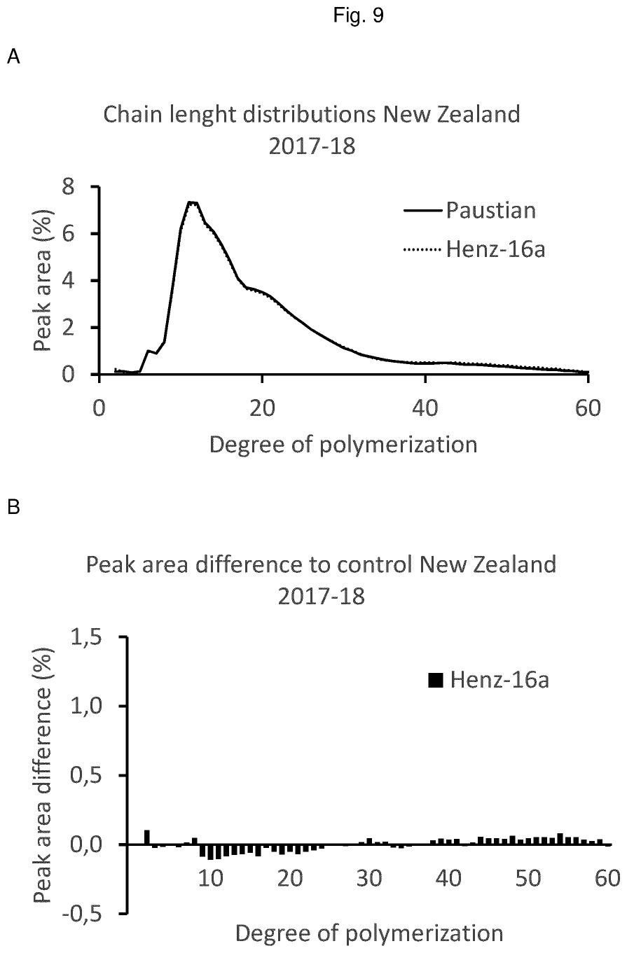

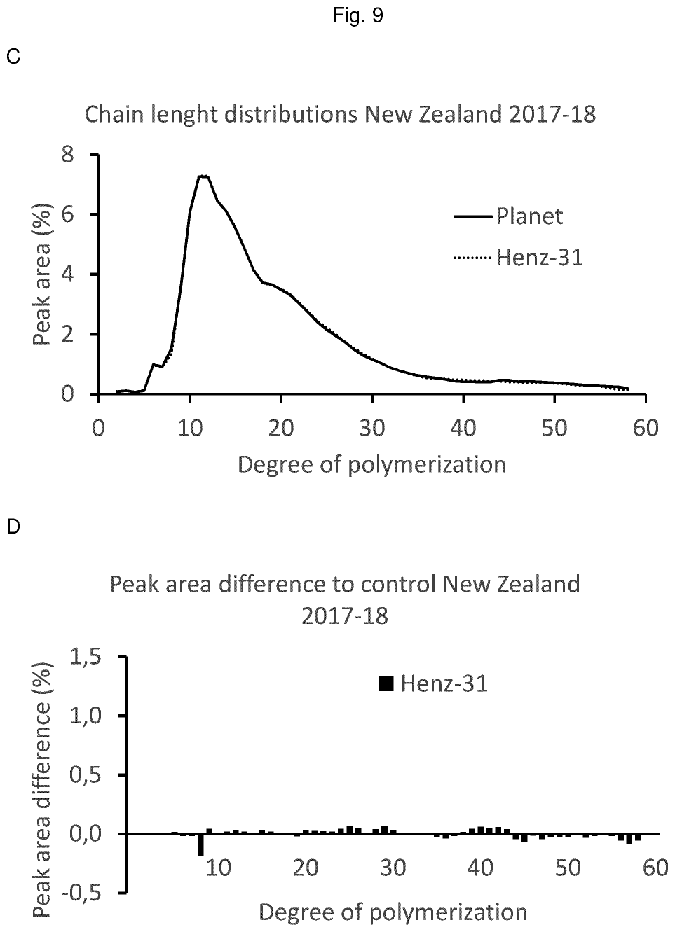

The degree of polymerization and the chain length distribution results are shown in FIGS. 8 and 9.

The chain length distribution profile of amylopectin of the mutants (HENZ-8, HENZ-9, HENZ-16, HENZ-18 and HENZ-31) were essentially identical to the two control barley plants: Planet and Paustian. The minor differences in chain length are a result of standard technical variation.

### Example 10. Yield and Grain Analysis

### Yield

Barley plants were grown in Denmark 2018 (2 rep) and New Zealand 2017/2018 (3-8 rep) and the yield of the barley plants were measured. The experimental results are summarized in Table 8.

TABLE 8

| | Yield | |
|---|---|---|
| | DK 2017 plots (2 reps) | NZ 2017/2018 plots (3-8 reps) |
| HENZ-16a | 5.30 | 1.64 |
| HENZ-18 | N/A | 1.71 |
| Paustian | 5.28 | 1.79 |
| HENZ-31 | 5.26 | 1.71 |
| Planet | 5.31 | 2.03 |

No significant difference between the mutated barley plants and the controls were observed.

Grain weight were analyzed from barley plants grown in New Zealand 2017/2018. The experimental results are summarized in Table 9.

TABLE 9

| | NZ 2017/2018 (10 technical reps) | | | |
|---|---|---|---|---|
| | Weight of | Average | | |
| | 1000 | grain | Grain | Diameter/length |
| | grains (g) | weight (mg) | diameter | ratio |
| HENZ-16a | 56.46 | 56.46 | 1.52 | 2.18 |
| HENZ-18 | 57.51 | 57.51 | 1.53 | 2.15 |
| Paustian | 55.11 | 55.11 | 1.48 | 2.11 |
| HENZ-31 | 62.20 | 62.20 | 1.59 | 2.30 |
| Planet | 62.64 | 62.64 | 1.58 | 2.30 |

### Protein, Water and Starch Content

The grain content of protein, water and starch was measured using a FOSS Infratec™ NOVA instrument according the manufactures instructions applying the calibration for these components provided by the supplier of the instrument. The results are shown in Table 10 below.

TABLE 10
New Zealand 2017/2018

| | Protein, % | Water, % | Starch, % |
|---|---|---|---|
| HENZ-16 | 11.9 ± 0.1 | 11.2 ± 0.0 | 62.3 ± 0.2 |
| HENZ-18 | 13.3 ± 0.0 | 11.3 ± 0.0 | 60.0 ± 0.2 |
| Paustian | 11.4 ± 0.1 | 11.1 ± 0.1 | 62.8 ± 0.1 |
| HENZ-31 | 10.8 ± 0.1 | 11.4 ± 0.1 | 63.4 ± 0.1 |
| Planet | 11.5 ± 0.2 | 11.2 ± 0.1 | 62.9 ± 0.2 |

The results demonstrate that there are no significant difference in protein content, water content, and starch content between HENZ-16, HENZ-18, Paustian, HENZ-31 and Planet.

1. A barley plant, or a part thereof, wherein said barley plant carries a mutation in the HvLDI gene, wherein said mutated HvLDI gene encodes a mutant HvLDI polypeptide, wherein the mutation is one of the following mutations
a. a missense mutation resulting in a change from a proline to a different amino acid in one or more loop regions of mutant HvLDI polypeptide, wherein the loop regions are selected from the group consisting of amino acids corresponding to position 25 to 44 and amino acids corresponding to position 56 to 62 and amino acids corresponding to position 77 to 78 and amino acids corresponding to position 91 to 111 and amino acids corresponding to position 124 to 147 of SEQ ID NO:1; or
b. a missense mutation resulting in a change from a negatively charged amino acid to a non-negatively charged amino acid in one or more alpha helix regions of mutant HvLDI polypeptide, wherein the alpha helix regions are selected from the group consisting of amino acids corresponding to position 45 to 55 and amino acids corresponding to position 63 to 76 and amino acids corresponding to position 79 to 90 and amino acids corresponding to position 112 to 123 of SEQ ID NO:1.

2. The barley plant or part thereof according claim 1, wherein said mutant HvLDI polypeptide is identical to the mature wt HvLDI polypeptide or natural variants thereof apart from the mutation in the specified position(s).

3. The barley plant or part thereof according to any one of the preceding claims, wherein the mutant HvLDI polypeptide comprises or consists of the amino acid sequence from position 25 to 142 of SEQ ID NO: 3 or from position 25 to 147 of SEQ ID NO: 3 or consists of the amino acid sequence from position 25 to 142 of SEQ ID NO: 4 or from position 25 to 147 of SEQ ID NO: 4.

4. The barley plant or part thereof according to any one of the preceding claims, wherein the mutant HvLDI polypeptide comprises or consists of the amino acid sequence from position 25 to 142 of SEQ ID NO: 6 or from position 25 to 147 of SEQ ID NO: 6.

5. The barley plant or part thereof according to any one of the preceding claims, wherein grains or germinated grains or malt from said barley plant have a free HvLD activity at least 20% higher compared to the free HvLD activity measured in grains of a barley plant carrying a HvLDI gene encoding a wt HvLDI polypeptide, but otherwise of the same genotype, when cultivated and prepared under the same conditions.

6. The barley plant or part thereof, wherein said barley plant carries one or more mutations in the HvLDI gene selected from the group consisting of:
i. a mutation of nucleotide C to T at the position corresponding to nucleotide 966 of the coding sequence of the HvLDI gene (SEQ ID NO:2); and
ii. a mutation of nucleotide C to T at the position corresponding to nucleotide 967 of the coding sequence of the HvLDI gene (SEQ ID NO:2); and
iii. a mutation of nucleotide C to T at the position corresponding to nucleotide 968 of the coding sequence of the HvLDI gene (SEQ ID NO:2); and
iv. a mutation of G to A at the position corresponding to nucleotide 990 of the coding sequence of the HvLDI gene (SEQ ID NO:2).

7. The barley plant according to any one of the preceding claims, wherein the grains of said barley plant have a thousand grain weight of at least 45 gram, such as at least 50 gram, such as at least 55 gram.

8. The barley plant according to any one of the preceding claims, wherein the grains of said barley plant have a starch content of at least 50%, such as at least 55%, such as at least 60%.

9. The barley plant according to any one of the preceding items, wherein the barley plant further comprises a mutation in one or more additional genes, selected from the group consisting of:
a. a mutation in the gene encoding LOX-1 resulting in a total loss of functional LOX-1;
b. a mutation in the gene encoding LOX-2 resulting in a total loss of functional LOX-2
c. a mutation in the gene encoding MMT resulting in a total loss of functional MMT
d. a mutation in the gene encoding CsIF6, wherein said mutant gene encodes mutant CsIF6 protein with reduced CsIF6 activity;
e. a mutation in the gene encoding HRT gene leading to a loss of HRT function;
f. a mutation in the gene encoding HBL12 gene leading to a loss of HBL function;
g. a mutation in the gene encoding WRKY38 gene leading to a loss of WRKY38 function; and
h. an ant mutation, for example a mutation in the Hvmyb10 gene.

10. A plant product comprising the barley plant or a part thereof according to any one of the preceding claims.

11. The plant product according to claim 10, wherein the plant product is selected from the group consisting of
a. malt prepared from grains of said barley plant;
b. aqueous extract, for example wort, prepared from grains of said barley plant and/or from malt comprising processed grain(s) of said barley plant; and
c. a beverage, for example beer, prepared from said barley plant of parts thereof.

12. A method of preparing malt, said method comprising the steps of
a. providing grains of a barley plant according to any one of claims 1 to 9;
b. steeping and germinating said grains under predetermined conditions;
c. optionally, drying said germinated grains.

13. The method according to claim 12, wherein the steeping and germination comprises the following steps:
a. incubating grains of a barley plant according to any one of claims 1 to 9 in an aqueous solution for a period of 5 to 10 h under aeration;
b. draining off the aqueous solution and subjecting the grains to an air rest for 8 to 16 h, preferably under aeration;
c. incubating the grains in an aqueous solution for 2-10 h under aeration; and
d. draining off the aqueous solution and subjecting the grain to a second air rest phase for 8 to 20 h, preferably under aeration while maintaining a temperature in the range of 20 to 28° C.
wherein the water content of the grains is at least 20% at any timepoint after step a.

14. The method according to claim 12 or 13, wherein said steeping and germination is performed for in the range of 48 and 72 hours and step c of claim 12 is omitted.

15. A method of preparing an aqueous extract, said method comprising the steps of
a. providing grains of a barley plant according to any one of claims 1 to 9 and/or malt produced according to claims 12 to 14;
b. preparing an aqueous extract of said grains and/or said malt, for example a wort.

16. The method according to claim 15, wherein said aqueous extract has at least 10% more glucose, fructose and/or maltotriose compared to an aqueous extract of barley plants carrying a HvLDI gene encoding a wt HvLDI polypeptide, but otherwise of the same genotype, when prepared under the same conditions.

17. A method of producing a beverage, said method comprising the steps of
a. providing grains of a barley plant according to any one of claims 1 to 9 and/or malt produced according to claims 12 to 14 and/or an aqueous extract produced according to the method of claim 15 or 16
b. processing said aqueous extract into a beverage.
